(19) United States
(12) Patent Application Publication
(10) Pub. No.: US 2023/0270940 A1
(43) Pub. Date: Aug. 31, 2023

(54) DOFETILIDE DRUG DOSING METHODS AND SYSTEMS AND PRESCRIPTION DRUG USE RELATED SOFTWARE

(71) Applicant: AltaThera Pharmaceuticals LLC, Chicago, IL (US)

(72) Inventors: Jodi Devlin, Chicago, IL (US); Brandon Ira Kashfian, Chicago, IL (US)

(21) Appl. No.: 18/121,980

(22) Filed: Mar. 15, 2023

Related U.S. Application Data
(63) Continuation-in-part of application No. 17/566,840, filed on Dec. 31, 2021, now Pat. No. 11,610,660.
Application No. 17/566,840 is a continuation-in-part of application No. 17/892,301, filed on Aug. 22, 2022.
Application No. 17/892,301 is a continuation-in-part of application No. 17/566,840, filed on Dec. 31, 2021, now Pat. No. 11,610,660.
(60) Provisional application No. 63/235,500, filed on Aug. 20, 2021.
(60) Provisional application No. 63/276,947, filed on Nov. 8, 2021.
(60) Provisional application No. 63/235,500, filed on Aug. 20, 2021.
(60) Provisional application No. 63/345,068, filed on May 24, 2022.
(60) Provisional application No. 63/344,154, filed on May 20, 2022.
(60) Provisional application No. 63/340,581, filed on May 11, 2022.
(60) Provisional application No. 63/334,267, filed on Apr. 25, 2022.
(60) Provisional application No. 63/331,905, filed on Apr. 18, 2022.
(60) Provisional application No. 63/276,947, filed on Nov. 8, 2021.
(60) Provisional application No. 63/235,500, filed on Aug. 20, 2021.
(60) Provisional application No. 63/276,947, filed on Nov. 8, 2021.

(51) Int. Cl. A61M 5/172; A61K 31/145; A61K 9/00; A61J 7/00

(52) U.S. Cl. CPC A61M 5/1723; A61K 31/145; A61K 9/0019; A61J 7/0053; A61K 9/0053; A61M 2205/3303; A61M 2230/06

(57) ABSTRACT

The disclosure relates to a method which includes i) prompting input of: a creatinine clearance (CrCl) of a subject; an amount of an oral or IV maintenance dosage of dofetilide for the subject; and optionally a projected start time for dofetilide infusion; and ii) executing computer executable instructions to derive from a set of rules a dofetilide dosing protocol for the subject based on the inputs. The disclosure also relates to systems and medical devices configured to perform one or more steps of the method, as well as a non-transitory computer-readable storage medium comprising computer-executable instructions or software stored thereon capable of performing one or more steps of the method.

### CROSS-REFERENCE TO RELATED APPLICATIONS

The present application is a Continuation-in-Part application of U.S. application Ser. No. 17/566,840, filed Dec. 31, 2021, which application relies on the disclosure of and claims priority to and the benefit of the filing date of U.S. Provisional Application No. 63/235,500, filed Aug. 20, 2021, and U.S. Provisional Application No. 63/276,947, filed Nov. 8, 2021, and the present application is a Continuation-in-Part application of U.S. application Ser. No. 17/892,301, filed Aug. 22, 2022, which relies on the disclosure of and claims priority to and the benefit of the filing dates of U.S. Provisional Application Nos. 63/235,500, filed Aug. 20, 2021, 63/276,947, filed Nov. 8, 2021, and 63/331,905, filed Apr. 18, 2022, the disclosures of which are hereby incorporated by reference herein in their entireties.

### FIELD OF THE INVENTION

The present disclosure is directed to the field of cardiovascular pharmaceutics, and more particularly to computer-implemented methods of determining antiarrhythmic drug dosing and administering antiarrhythmic drugs for the treatment of cardiovascular conditions such as arrhythmias, and medical devices and systems capable of performing the methods.

### BACKGROUND

Determining the appropriate dosing of antiarrhythmic drugs can be complex, difficult to understand or apply, and/or time consuming. In situations where a patient is having a cardiovascular crisis, practitioners need to act quickly and, thus, make decisions quickly. Dosing an antiarrhythmic drug, however, often involves simultaneous and careful consideration of a plethora of parameters. Typical dosing parameters can include whether to administer the drug to a patient based on the patient's QT interval or QTc and/or the patient's capability of clearing the drug from their body (such as renal clearance as measured by creatinine clearance rate), how much drug to administer intravenously (loading dose) and for what amount of time, whether to administer multiple IV doses or a single IV dose, when to start oral and/or IV maintenance dosing of the drug relative to the start or completion of the IV loading dose, the amount of the target oral or IV maintenance dose for the patient, whether the patient is being initiated on the drug or whether the patient's current oral or IV maintenance drug therapy is being escalated to a higher oral or IV maintenance drug therapy, and the timing interval between subsequent doses (IV or oral maintenance doses). In some cases, one or more or all of these factors are considered in determining the dosing protocol for a patient that will be used for administering the antiarrhythmic drug to the patient. Software, such as a mobile application or app, appropriately programmed, can be used to determine and/or confirm a practitioner's determination of the dosing protocol appropriate for a particular patient based on one or more or all of the above parameters, or other relevant parameters. The systematic application of known dosing criteria by a software app, when applied to the circumstances of a particular patient, can increase the safety of dosing/administering antiarrhythmic drugs, such as sotalol, dofetilide, amiodarone, ibutilide, dronedarone, procainamide, flecainide, or propafenone.

Sotalol is an antiarrhythmic drug with Class II (beta-adrenoreceptor blocking) and Class III (cardiac action potential duration prolongation) properties. Sotalol is indicated for the maintenance of normal sinus rhythm (delay in time to recurrence of atrial fibrillation/atrial flutter (AFIB/AFL)) in patients with symptomatic AFIB/AFL who are currently in sinus rhythm, and indicated for the treatment of life-threatening ventricular tachycardia.

Typical adult dosages for oral sotalol are 80 mg, 120 mg, and 160 mg, while intravenous doses of sotalol range from 60-112.5 mg.

Sotalol hydrochloride is a white, crystalline solid with a molecular weight of 308.8. It is hydrophilic, soluble in water, propylene glycol and ethanol, but is only slightly soluble in chloroform. Chemically, sotalol hydrochloride is d,1-N-[4-[1-hydroxy-2-[(1-methyl ethyl) amino]ethyl]phenyl]methane-sulfonamide monohydrochloride. The molecular formula is Cl₂H₂O N₂O₃S·HCl. Sotalol is represented by the following structural formula:

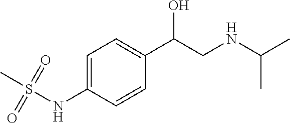 [SMILES: CC(C)NCC(O)c1ccc(NS(C)(=O)=O)cc1]

Sotalol can cause serious ventricular arrhythmias, primarily Torsade de Pointes (TdP) type ventricular tachycardia, a polymorphic ventricular tachycardia associated with QTc prolongation. QTc prolongation is directly related to the concentration of sotalol in the patient. As a result, the US FDA has mandated in-hospital QTc monitoring for at least three days upon initial sotalol hydrochloride loading and for dose escalation. Sotalol is currently approved in the US for oral administration (for example, under the brand name BETAPACE AF®, Bayer HealthCare Pharmaceuticals Inc.) and is approved for IV administration (AltaThera Pharmaceuticals LLC).

Ibutilide as an antiarrhythmic drug is characterized by predominantly Class III properties and is represented by the following structural formula:

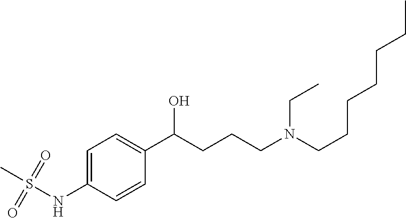 [SMILES: CCCCCCCN(CC)CCCC(O)c1ccc(NS(C)(=O)=O)cc1]

Ibutilide (a methanesulfonanilide derivative) is currently approved in the United States for intravenous administration (under the brand name CORVERT®, Pfizer Inc.) for the treatment of atrial fibrillation or atrial flutter. Patients with atrial arrhythmias of recent onset are more likely to respond to ibutilide. Typical adult IV doses of ibutilide are in the range of about 1-2 mg.

Another antiarrhythmic, dofetilide, is a Class III antiarrhythmic agent represented by the following structural formula:

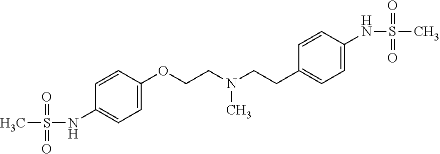

The mechanism of action for dofetilide is through blocking cardiac ion channels of the rapid component of the delayed rectifier potassium current Ikr. The agent, a sulfonamide, is approved to treat atrial fibrillation and atrial flutter. Dofetilide may additionally be useful for hospitalized people with atrial fibrillation cardioverted to normal sinus rhythm, without ventricular arrhythmias or various forms of blocks, in patients with normal kidney function. Dofetilide normalizes sinus rhythm by prolonging cardiac action potential duration and effective refractory period due to delayed repolarization without affecting conduction velocity. Dofetilide is currently approved in the United States for oral administration (under the brand name TIKOSYN®, Pfizer Inc.). Typical oral doses of dofetilide are in the range of 125-500 mcg (twice daily), while intravenous doses can be in the range of about 0.1 mcg/kg to 3 mcg/kg or more.

Amiodarone (for example, amiodarone hydrochloride: 2-butyl-3-benzafuranyl)[4-[2-(diethylamino)ethoxy]-3,5-diiodophenyl]-methanone hydrochloride) is also a Class III antiarrhythmic drug and is represented by the following structural formula:

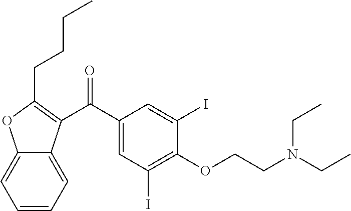 [SMILES: CCCCc1oc2ccccc2c1C(=O)c1cc(I)c(OCCN(CC)CC)c(I)c1]

Amiodarone is used for the treatment of life-threatening recurrent VF or life-threatening recurrent hemodynamically unstable VT and is usually used for patients who are intolerant to or who may not respond adequately to other antiarrhythmic drugs. It is currently approved in the United States for both oral and intravenous administration and is marketed under brand names including CORDARONE®, PACERONE®, and NEXTERONE®. Typical doses for oral amiodarone are in the range of 100-400 mg, with a loading dose of 800-1600 mg/day and a maintenance dose of 600-800 mg/day tapering off to 400 mg/day, while intravenous dosing of amiodarone is in the range of 150-540 mg.

Dronedarone (400 mg tablets, typically administered twice daily) is used for the treatment of paroxysmal or persistent atrial fibrillation or atrial flutter and can be used with patients who have experienced a recent AF/AFL episode who are in sinus rhythm or who will be cardioverted. Dronedarone is marketed under the brand name MULTAQ® (Sanofi Aventis) and has the following structural formula:

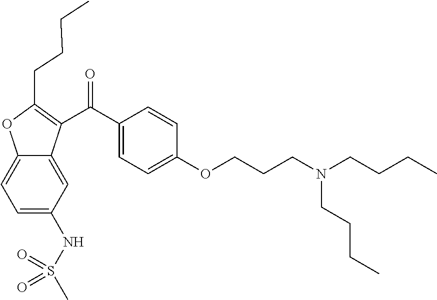 [SMILES: CCCCc1oc2ccc(NS(C)(=O)=O)cc2c1C(=O)c1ccc(OCCCN(CCCC)CCCC)cc1]

Flecainide is a Class IC drug used for the treatment of ventricular tachycardia, atrial fibrillation, atrial flutter, Wolff-Parkinson-White syndrome and paroxysmal supraventricular tachycardia and is represented by the following structural formula:

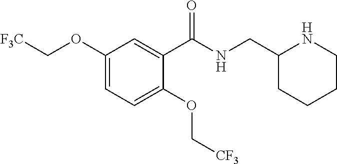 [SMILES: O=C(NCC1CCCCN1)c1cc(OCC(F)(F)F)ccc1OCC(F)(F)F]

Flecainide is typically administered to adults starting with 50-100 mg (twice a day) with the maximum recommended dose being 400 mg/day and is sold under the brand name TAMBOCOR® (3M Pharmaceuticals). Renal and liver adjustments may be needed in some cases.

Procainamide is typically administered in oral doses of 1500-2500 mg (twice daily) to adults and for intravenous doses in the range of 15-18 mg/kg or 100 mg, and up to a total of 1 gram/day. For patients with low renal or liver clearance abilities, adjustments are recommended. Procainamide is marketed under the brand names including PRONESTYL®, PRONESTYL-SR®, PROCAN SR®, and PROCANBID®.

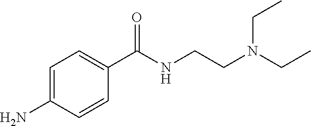 [SMILES: CCN(CC)CCNC(=O)c1ccc(N)cc1]

Propafenone (RHTHMOL®, RYTHMOL SR®) is used for the treatment of atrial fibrillation, atrial flutter, ventricular tachycardia and paroxysmal supraventricular tachycardia. A typical adult oral dose is in the range of about 150-425 mg, such as 150 mg at 8 hour intervals with a maximum dose of 800 mg/day. Adjustments, such as dose reduction, may be needed for patients with renal or liver clearance difficulty.

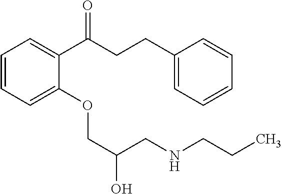 [SMILES: CCCNCC(O)COc1ccccc1C(=O)CCc1ccccc1]

More specific dosing information, mechanisms of action, contraindications, adverse effects, drug interactions, if any, renal and/or liver clearance thresholds, if applicable, etc. for various antiarrhythmic drugs can be found in the following product labels: (1) Amiodarone HCl injection for intravenous use [package insert], Lake Forest, Ill.: Hospira, Inc.; Initial U.S. Approval: 1995; (2) Cordarone® (amiodarone HCl) Tablets [package insert], Philadelphia, Pa.: Wyeth Pharmaceuticals Inc.; 2004; (3) TIKOSYN® (dofetilide) Capsules [package insert], NY, N.Y.: Pfizer Inc.; 2014; (4) MULTAQ (dronedarone) tablets, for oral use [package insert], Bridgewater, N.J.: Sanofi-Aventis U.S. LLC; 2020; (5) FLECAINIDE ACETATE TABLETS, USP [package insert], Jacksonville, Fla.: Ranbaxy Pharmaceuticals Inc.; 2003; (6) IBUTILIDE FUMARATE INJECTION [package insert], Morgantown, W. Va.: Mylan Institutional LLC; 2020; (7) PROCAINAMIDE HYDROCHLORIDE Injection, USP [package insert], Lake Forest, Ill.: Hospira, Inc.; 2021; (8) RYTHMOL (propafenone hydrochloride tablets), for oral use [package insert], Research Triangle Park, N.C.: GlaxoSmithKline; 2018. Each of these product labels is hereby incorporated by reference herein in its entirety.

### SUMMARY

Described herein are methods of administering one or more antiarrhythmic drug, such as a Class I, Class II or Class III antiarrhythmic drug, such as sotalol, dofetilide, amiodarone, ibutilide, dronedarone, procainamide, flecainide, or propafenone, to a patient in need thereof in an amount effective for treating a cardiovascular condition of the patient, such as atrial fibrillation (AFIB); atrial flutter (AF or AFL); ventricular tachycardia; hemodynamically stable or unstable ventricular tachycardia (VT); ventricular fibrillation (VF); paroxysmal supraventricular tachycardia; paroxysmal atrial fibrillation; heart failure; coronary artery disease; pulmonary artery hypertension; maintenance of normal sinus rhythm (delay in time to recurrence of atrial fibrillation/atrial flutter (AFIB/AFL)) for example in patients with symptomatic AFIB/AFL and/or who are currently in sinus rhythm, and/or indicated for the treatment of life-threatening ventricular tachycardia; and/or treating people, such as hospitalized people, with atrial fibrillation cardioverted to normal sinus rhythm, without ventricular arrhythmias or various forms of blocks, in patients with normal kidney function; treating life-threatening recurrent VF or life-threatening recurrent hemodynamically unstable VT; treating paroxysmal or persistent atrial fibrillation or atrial flutter, such as with patients who have experienced a recent AF/AFL episode who are in sinus rhythm or who will be cardioverted; and/or treating Wolff-Parkinson-White syndrome and/or paroxysmal supraventricular tachycardia.

In one implementation, a method of administering an antiarrhythmic drug includes i) prompting input relating to one or more health characteristics of a patient and relating to one or more antiarrhythmic drug (oral and/or IV) dosing characteristics/history of the patient, including one or more or any combination of patient's age, patient's weight, sex of patient, patient's heart rate, patient's blood pressure, patient's temperature, patient's creatinine clearance, patient's renal or liver function, patient's cardiovascular condition, patient's medications, amount of current and/or previous and/or target oral or IV maintenance dose, time from or time of last oral or IV maintenance dose, current or previous antiarrhythmic drug administered, and/or desired/target antiarrhythmic drug to be administered or switched to;

ii) determining drug appropriateness for the patient, an amount of an IV loading dose, a time for administering of (or any minimum delay from completion of the IV loading dose to) a first oral or IV maintenance dose, and/or an oral or IV maintenance dosing interval based on the input, a target oral maintenance dose, whether the patient is being initiated or escalated on the drug, and outputting a drug dosing protocol for the patient based on the drug appropriateness for the patient, the patient's creatinine clearance, the patient's renal or liver function, the patient's cardiovascular condition, the patient's medications, the amount of the IV loading dose, a time for administering of (or any minimum delay to) the first oral dose, the oral dosing interval, and the target amount of the maintenance oral dose based on the determining.

Embodiments include a method of administering sotalol, such as sotalol hydrochloride, the method comprising: i) prompting input relating to one or more health characteristics of a patient and sotalol oral and/or IV dosing characteristics/history of the patient, including one or more or any combination of patient's QT interval or QTc, patient's creatinine clearance, amount of current and/or previous and/or target oral or IV sotalol maintenance dose, projected start time for infusion, whether the patient is being initiated on or escalated to a higher oral dose of sotalol hydrochloride, time from or time of last oral sotalol maintenance dose, current or previous antiarrhythmic drug administered;

ii) determining sotalol appropriateness for the patient, an amount of an IV sotalol loading dose, a time for administering of (or any minimum delay from completion of the IV loading dose to) a first sotalol oral or IV maintenance dose, and/or oral or IV dosing interval between sotalol maintenance doses based on the input, a target oral or IV sotalol maintenance dose, and outputting a sotalol dosing protocol for the patient based on any one or more or all of the sotalol appropriateness for the patient, the patient's creatinine clearance, the patient's renal function, the patient's cardiovascular condition, the IV sotalol loading dose, a time for administering of a first oral sotalol maintenance dose (or any minimum delay to the first sotalol oral or IV maintenance dose from completion of the IV loading dose), the sotalol oral or IV maintenance dosing interval, and the target amount of the maintenance sotalol oral or IV dose based on the determining.

Embodiments include a method of administering dofetilide, the method comprising: i) prompting input relating to one or more health characteristics of a patient and dofetilide oral and/or IV dosing characteristics/history of the patient, including one or more or any combination of patient's age, patient's weight, sex of patient, patient's heart rate, patient's blood pressure, patient's temperature, patient's creatinine clearance, patient's renal function, patient's cardiovascular condition, patient's medications, amount of current and/or previous and/or target oral or IV dofetilide maintenance dose, a projected start time for infusion, whether the patient is being initiated on or escalated to a higher oral dofetilide dose, time from or time of last oral or IV dofetilide maintenance dose, current or previous antiarrhythmic drug administered;

ii) determining any one or more of or all of dofetilide appropriateness for the patient, an amount of an IV dofetilide loading dose, a time for administering of (or any minimum delay to) a first dofetilide oral or IV maintenance dose, and/or oral or IV dosing interval between maintenance dofetilide doses based on the input, an amount of a target oral or IV dofetilide maintenance dose, and outputting a dofetilide dosing protocol for the patient based on any one or more of or all of the dofetilide appropriateness for the patient, the patient's creatinine clearance, the patient's renal function, the patient's cardiovascular condition, the patient's medications, the amount of the IV dofetilide loading dose or start time for infusion, a time for administering of (or any minimum delay to) the first dofetilide oral or IV maintenance dose, the dofetilide oral or IV maintenance dosing interval, and the target amount of the maintenance dofetilide oral or IV dose based on the determining.

The methods can be effectuated through software steps performed by a computer processor and other computer components. Additional implementations include as components of the system one or more of a computer, computing device, or medical device, such as an EKG measuring device, and/or combinations thereof forming a system, designed or configured and connected together in a manner to communicate with and/or control components of the system to perform one or more steps of the method implementations. Additional implementations include a non-transitory computer-readable storage medium comprising computer-readable code, computer-readable instructions, computer-executable instructions, or “software” stored thereon capable of performing one or more steps of the method implementations. More specific implementations of the preceding and their features are provided in the Drawings and the Detailed Description.

According to some advantages, the implementations provide an intravenous loading dose for sotalol, dofetilide, amiodarone, ibutilide, dronedarone, procainamide, flecainide, and/or propafenone that is expected to achieve steady state concentration faster compared to conventional oral dosing for these antiarrhythmics. As such, the implementations can reduce the length of hospitalization and QTc monitoring required in comparison to conventional oral dosing, and allow a health care provider to safely and efficaciously administer one or more antiarrhythmic drug, or switch from one antiarrhythmic drug to another, in a manner which minimizes the potential for concentration-dependent adverse effects such as ventricular arrhythmias (e.g. Torsade de Pointes).

### More Specific Aspects of Embodiments of the Invention Include

Aspect A, which is a treatment management application configured to perform a set of operations comprising:

(i) reading, prompting input of and/or accepting the following as inputs:

a projected start time for a dofetilide infusion, such as a 1 hour infusion;

a creatinine clearance (CrCl) or creatinine level of a subject; and

an amount of an oral or IV target/maintenance dosage of dofetilide for the subject; and

(ii) executing computer executable instructions to derive from a set of rules a dofetilide dosing protocol, such as an IV and oral dosing protocol or an IV and IV dosing protocol, for the subject based on the inputs, the dosing protocol comprising:

one or more amount of the dofetilide for infusion as a loading dose;

a time for a first maintenance (IV or oral) dose of dofetilide; and

optionally a time for a second maintenance (IV or oral) oral dose of dofetilide; and

(iii) outputting the dofetilide dosing protocol.

Aspect B is the treatment management application of Aspect A, wherein the set of operations further comprises reading, prompting input of and/or accepting as an input an actual start time for the dofetilide infusion and outputting a revised dosing protocol with confirmed or updated times for the first maintenance dose and optionally the second maintenance dose.

Aspect C is the treatment management application of Aspect A or B, wherein 500 mcg is the amount of dofetilide for one or more of the maintenance doses and the amount of dofetilide for infusion as the loading dose is in the range of 250-750 mcg.

Aspect D is the treatment management application of any of Aspects A-C, wherein 250 mcg is the amount of dofetilide for one or more of the maintenance doses and the amount of dofetilide for infusion as the loading dose is in the range of 125-375 mcg.

Aspect E is the treatment management application of any of Aspects A-D, wherein 125 mcg is the amount of dofetilide for one or more of the maintenance doses and the amount of dofetilide for infusion as the loading dose is in the range of 62.5-187.5 mcg.

Aspect F is the treatment management application of any of Aspects A-G, wherein 500 mcg is the amount of dofetilide for one or more of the maintenance doses and the amount of dofetilide for infusion as the loading dose is in the range of 125-625 mcg.

Aspect G is the treatment management application of any of Aspects A-F, wherein 250 mcg is the amount of dofetilide for one or more of the maintenance doses and the amount of dofetilide for infusion as the loading dose is in the range of 62.5-312.5 mcg.

Aspect H is the treatment management application of any of Aspects A-G, wherein 125 mcg is the amount of dofetilide for one or more of the maintenance doses and the amount of dofetilide for infusion as the loading dose is in the range of 31.25-156.25 mcg.

Aspect I is the treatment management application of any of Aspects A-H, wherein the amount for the infusion of dofetilide as the loading dose is higher than 3 μg/kg.

Aspect J is the treatment management application of any of Aspects A-H, wherein the amount for the infusion of dofetilide as the loading dose is in the range of 1.6-3 μg/kg.

Aspect K is the treatment management application of any of Aspects A-H, wherein the amount for the infusion of dofetilide as the loading dose is in the range of 0.8-1.6 μg/kg.

Aspect L is the treatment management application of any of Aspects A-H, wherein the amount for the infusion of dofetilide as the loading dose is in the range of 0.1-0.8 μg/kg.

Aspect M is the treatment management application of any of Aspects A-L, wherein the second maintenance dose (IV or oral) is a higher dose than the first maintenance (IV or oral) dose.

Aspect N is the treatment management application of any of Aspects A-L, wherein the second maintenance dose (IV or oral) is a lower dose than the first maintenance (IV or oral) dose.

Aspect O is the treatment management application of any of Aspects A-N, wherein the amount of the dofetilide for infusion as a loading dose is an amount in the range of about ±50% of the amount of one or more of the maintenance dosages of dofetilide.

Aspect P is the treatment management application of any of Aspects A-N, wherein the amount of the dofetilide for infusion as a loading dose is an amount in the range of about ±25% of the amount of one or more of the maintenance dosages of dofetilide.

Aspect Q is the treatment management application of any of Aspects A-P, wherein the patient has a creatinine clearance of 20-60 mL/min.

Aspect R is the treatment management application of any of Aspects A-P, wherein the creatinine clearance of the patient is >60 mL/min.

Aspect S is a medical device comprising: an electronic device, optionally a handheld electronic device; software stored on or accessible to the electronic device and operably configured to: (i) read, prompt input of and/or accept as inputs: a creatinine clearance (CrCl) of a subject; an amount of a dofetilide maintenance dosage (IV or oral) for the subject; and optionally a projected or actual start time for dofetilide infusion; and (ii) execute computer executable instructions to derive from a set of rules a dofetilide dosing protocol (IV and oral protocol or IV loading and IV maintenance protocol) for the subject based on one or more of or all of the inputs, the dosing protocol comprising at least one or more amount of the dofetilide for infusion as a loading dose and a time for a first maintenance (IV or oral) dose of dofetilide based on the projected or actual start time; and (iii) output the dofetilide dosing protocol; and a display capable of displaying the output of the dosing protocol to a user.

Aspect T is a method of treating a subject comprising: administering dofetilide to a subject based on outputs from a treatment management application configured to perform a set of operations comprising: (i) reading, prompting input of and/or accepting the following as inputs: a projected start time for a dofetilide infusion, such as a 1 hour infusion; a creatinine clearance (CrCl) or creatinine level of a subject; and an amount of a maintenance dose (IV or oral) dofetilide for the subject; and (ii) executing computer executable instructions to derive from a set of rules a dofetilide dosing protocol (IV and oral protocol or IV loading and IV maintenance protocol) for the subject based on the inputs, the dosing protocol comprising: one or more amount of the dofetilide for infusion as a loading dose; a time for a first maintenance (IV or oral) dose of dofetilide; and optionally a time for a second maintenance (IV or oral) dose of dofetilide; and (iii) outputting the dofetilide dosing protocol; and optionally administering the dofetilide in a manner for providing for maintenance of normal sinus rhythm in patients with symptomatic atrial fibrillation or atrial flutter and/or who are currently in sinus rhythm.

Aspect 1: A method comprising: i) prompting input of a baseline QTc value and optionally one or more additional health characteristics of a patient; ii) determining whether the inputted baseline QTc value exceeds a threshold QTc value, and, based on the determining: a) if the inputted baseline QTc value exceeds the threshold QTc value, then outputting a message that one or more antiarrhythmic drug, such as dofetilide, is not recommended for the patient, or b) if the inputted baseline QTc value does not exceed the threshold QTc value, then: iii) prompting input of a creatinine clearance, any current antiarrhythmic drug oral or IV maintenance dose, and target antiarrhythmic drug oral or IV maintenance dose of a patient, including whether the patient is being initiated on the drug or escalated to a higher oral dose; iv) determining an IV loading dose, a time for administering of or any minimum delay to a first oral or IV maintenance dose, and/or an oral or IV maintenance dosing interval based on the inputted creatinine clearance, current oral or IV dose, whether the patient is being initiated or escalated, and the target oral or IV dose; and v) outputting the determined IV loading dose, a time for administering or any minimum delay to first oral or IV maintenance dose, and/or oral or IV dosing interval.

Aspect 2: A method comprising: i) prompting input of one or more health characteristics of a patient and one or more current or previous antiarrhythmic oral or IV dosing characteristics of the patient; ii) determining appropriateness of the antiarrhythmic drug for the patient, an IV loading dose, any minimum delay to first oral or IV maintenance dose, and/or oral or IV maintenance dosing interval based on the input; iii) outputting a message regarding the drug appropriateness for the patient, IV loading dose, any minimum delay to first oral or IV maintenance dose, and/or oral or IV maintenance dosing interval based on the determining.

Aspect 3: The method of Aspect 1 or 2, wherein the health characteristics of the patient comprise information that can affect one or more pharmacokinetic characteristics or behaviors of the antiarrhythmic drug in the patient.

Aspect 4: The method of any Aspects 1-3, wherein the one or more pharmacokinetic characteristics or behaviors of the antiarrhythmic drug in the patient comprise one or more of clearance, half-life, bioavailability, absorption, Cₘₐₓ, Tₘₐₓ, and/or Area under the Curve (AUC).

Aspect 5: The method of any of the above Aspects, wherein the health characteristics comprise any one or more of age, weight, sex or renal function of the patient.

Aspect 6: The method of Aspect 5, wherein the patient renal function is measured by patient creatinine clearance.

Aspect 7: The method of any of the above Aspects, wherein the health characteristics comprise concomitant medications.

Aspect 8: The method of any of the above Aspects, wherein the health characteristics comprise information indicating a potential risk of an adverse effect of the antiarrhythmic drug.

Aspect 9: The method of any of the above Aspects, wherein the adverse effect comprises one or more of proarrhythmia (e.g. Torsade de Pointes (TdP)), bradycardia, sinus pauses or sinus arrest, hypotension, heart failure, negative inotropy, dyspnea, and/or fatigue, or any other adverse effect.

Aspect 10: The method of any of the above Aspects, wherein the information indicating the potential risk of the antiarrhythmic drug adverse effect comprises patient QT interval or QTc.

Aspect 11: The method of any of the above Aspects, wherein the information indicating the potential risk of the antiarrhythmic drug adverse effect comprises concomitant medications.

Aspect 12: The method of any of the above Aspects, wherein the health characteristics comprise information relating to appropriateness of initiation or continuation of the antiarrhythmic drug for that patient.

Aspect 13: The method of any of the above Aspects, wherein the one or more antiarrhythmic drug oral or IV maintenance dosing characteristics comprise a current, previous and/or target patient oral or IV maintenance dose of the antiarrhythmic drug.

Aspect 14: The method of any of the above Aspects, wherein the prompting input step is performed through one or more input-output (I/O) interface on a computer, computing device, or medical device.

Aspect 15: The method of any of the above Aspects, wherein the calculating and determining steps are performed by way of a processor coupled with a memory on a computer, computing device, or medical device which has various IV loading doses, minimum delays to first oral or IV maintenance dose, and/or oral or IV maintenance dosing intervals of the antiarrhythmic drug stored within.

Aspect 16: The method of any of the above Aspects, wherein the IV loading doses, minimum delays to first oral or IV maintenance dose, and/or oral or IV maintenance dosing intervals are stored in a relational database in the memory.

Aspect 17: The method of any of the above Aspects, wherein the IV loading dose(s), minimum delay(s) to first oral or IV maintenance dose, and/or oral or IV maintenance dosing interval(s) are represented in Table 1.

Aspect 18: The method of any of the above Aspects, wherein the IV loading dose(s), minimum delay(s) to first oral dose, and/or oral or IV maintenance dosing interval(s) are stratified according to tiers of creatinine clearance ranges or values and/or or drug initiation or escalation values, such as those represented in Table 1.

Aspect 19: The method of any of the above Aspects, further comprising: i) determining one or more IV loading dose, any minimum delay to first oral or IV maintenance dose, and/or oral or IV maintenance dosing intervals from computer-based simulations incorporating dose-exposure-QTc relationships for the antiarrhythmic drug, such as dofetilide; ii) storing the determined IV loading dose, any minimum delay to first oral or IV maintenance dose, and/or oral or IV maintenance dosing intervals in a memory on a computer, computing device, or medical device, such as in a relational database; and iii) retrieving one or more stored drug IV loading dose, any minimum delay to first oral or IV maintenance dose, and/or oral or IV maintenance dosing interval values based on one or more of the inputted patient health characteristics and/or drug dosing characteristics.

Aspect 20: The method of any of the above Aspects, wherein the calculating steps are performed by way of one or more mathematical operations programmed on the computer, computing device, or medical device such as multiplication, addition, subtraction, division, logarithmic functions, exponential functions, and/or algorithmic functions.

Aspect 21: The method of any of the above Aspects, wherein the determining steps are performed by retrieving a corresponding IV loading dose, any minimum delay to first oral or IV maintenance dose, and/or oral or IV maintenance dosing interval stored in a database within the memory of the computer, computing device, or medical device based on one or more of the patient health characteristics or drug dosing characteristics inputted.

Aspect 22: The method of any of the above Aspects, wherein the retrieving comprises matching one or more inputted characteristics with values or ranges used to stratify drug IV loading dose, any minimum delay to first oral or IV maintenance dose, and/or oral or IV maintenance dosing interval values.

Aspect 23: The method of any of the above Aspects, wherein the outputting step is performed by way of a visual output device or an auditory output device on a computer, computing device, or medical device.

Aspect 24: The method of any of the above Aspects, wherein the steps of the method are performed on a single computer, computing device, or medical device.

Aspect 25: The method of any of the above Aspects, wherein the steps of the method are performed on a computer system or network comprising multiple computers, computing devices, or medical devices.

Aspect 26: The method of any of the above Aspects, wherein the medical device is capable of measuring patient EKG, such as a 12-lead EKG or a Holter monitor, and one or more steps of the method are performed on the EKG measuring device.

Aspect 27: The method of any of the above Aspects, wherein some steps of the method are performed on the EKG measuring device, and some steps of the method are performed on a computer or computing device.

Aspect 28: The method of any of the above Aspects, wherein all steps of the method are performed on the EKG measuring device.

Aspect 29: A computer, computing device, or medical device, such as an EKG measuring device, designed or configured to perform one or more steps of the method of any of the above Aspects.

Aspect 30: A non-transitory computer-readable storage medium comprising computer-readable code, computer-readable instructions, computer-executable instructions, or software stored thereon capable of performing one or more steps of the method of any of the above Aspects.

Aspect 31: The non-transitory computer-readable storage medium of any of the above Aspects, further comprising data comprising drug IV loading dose, minimum delay to first oral or IV maintenance dose, and/or oral or IV maintenance dosing interval stratified according to tiers of creatinine clearance ranges and/or or initiation or escalation values.

Aspect 32: The non-transitory computer-readable storage medium of any of the above Aspects, wherein the data relates to dofetilide administration.

Aspect 33: The non-transitory computer-readable storage medium of any of the above Aspects, wherein the data is stored in a relational database.

Aspect 34: A method comprising: i) determining one or more antiarrhythmic drug IV loading dose, minimum delay (if any) to first oral or IV maintenance dose, and/or oral or IV maintenance dosing intervals from computer-based simulations incorporating antiarrhythmic drug dose-exposure-QTc relationships; and ii) storing the determined IV loading dose, minimum delay (if any) to first oral or IV maintenance dose, and/or oral or IV maintenance dosing intervals on a non-transitory computer readable storage medium, such as the non-transitory computer-readable storage medium of any of the above Aspects.

Aspect 35: The method, computer, computing device, medical device, or non-transitory computer-readable storage medium of any preceding Aspect, wherein the drug dose-exposure-QTc relationships are represented within the computer-based simulations by one or more mathematical functions, equations, or algorithms.

Aspect 36: A system for controlling administration of one or more antiarrhythmic drug to a patient, comprising: i) an input/output interface configured to accept and display patient data obtained from user input and/or one or more monitoring device configured to monitor one or more physiological measurement of a patient; ii) a data storage module configured to store the patient data, the one or more physiological measurement of the patient and one or more dosing criteria for administering one or more antiarrhythmic drug to the patient; and iii) one or more control module comprising one or more processor for executing computer-readable instructions for: a) determining an intravenous dose of the antiarrhythmic drug based on one or more of the patient data, one or more of the physiological measurement of the patient, and the dosing criteria; and b) providing instructions for controlling and/or controlling an infusion pump to administer the intravenous dose to the patient. In embodiments, the dosing criteria can be provided from labeling instructions for any antiarrhythmic drug, including those provided in any of the product labels referenced herein.

Aspect 37: A system comprising: i) an input/output interface; ii) a memory configured to store dosing criteria for one or more antiarrhythmic drug and store data relating to a patient; iii) a computer processor operatively connected to the memory configured to analyze the data relating to the patient and identify one or more acceptable IV dose of the antiarrhythmic drug.

Aspect 38: A system comprising: i) an input/output interface; ii) a medical monitoring device configured to monitor one or more physiological measurement of a patient; iii) an infusion pump for administering one or more antiarrhythmic drug to a patient; iv) one or more control module in communication with the medical device and the infusion pump and comprising computer-executable instructions capable of performing the method of any of the above Aspects by receiving data relating to the patient from the medical device, determining a dosing protocol for the patient based on the data relating to the patient and dosing criteria for the antiarrhythmic drug, and instructing the infusion pump to deliver antiarrhythmic drug to the patient according to the dosing protocol.

Aspect 39: A treatment management application configured to run on a mobile device, the treatment management application comprising: i) a first interface configured to accept patient data, optionally from a patient monitor; ii) a second interface invoked in response to submission of the patient data and configured to provide instructions (e.g., computer-executable instructions) for performing a treatment protocol; wherein the treatment management application is configured to perform operations comprising: a) retrieving the patient data; b) accessing drug dosing criteria from the application, a database, a server, the internet or a computer network; c) deriving the treatment protocol based on the patient data and the drug dosing criteria; and d) reporting, displaying or causing execution of the instructions for performing the treatment protocol, optionally causing execution of the instructions by a control module and/or an infusion pump for administering drugs intravenously.

Aspect 40: A system for administering a drug into a body of a patient with the aid of a computer-controlled dosage device, the system comprising: i) one or more patient monitor configured to obtain one or more physiological measurement of the patient; ii) an input/output interface; iii) a processing module configured to perform the following: a) retrieve patient data and the physiological measurements of the patient; b) access stored drug dosing criteria; c) report or display a treatment plan based on the patient data, the physiological measurements of the patient, and the drug dosage criteria; and iii) a control module configured to accept the treatment plan from the processing module and transmit the treatment plan to a computer-controlled dosage device; wherein the computer-controlled dosage device is capable of administering the drug into the body of the patient using an infusion pump according to the treatment plan.

Aspect 41: The system any of the above Aspects, wherein the one or more physiological measurement is chosen from QT interval, QTc, blood pressure, heart rate, or combinations thereof.

Aspect 42: The method of any of the above Aspects, wherein the antiarrhythmic drug is one or more of sotalol, dofetilide, amiodarone, ibutilide, dronedarone, procainamide, flecainide, or propafenone.

Aspect 43: A treatment management application configured to manage treatment of a patient according to the method of any of the Aspects disclosed herein.

Aspect 44: A system for administering a drug into a body of a patient according to any of the methods of any of the Aspects disclosed herein.

Aspect 45: The system or application of any of the Aspects disclosed herein, wherein the patient data comprises one or more of patient age, patient weight, patient sex, patient renal function, such as creatinine clearance (CrCl), patient medication(s), patient heart rate, patient blood pressure, patient temperature, and/or patient cardiovascular condition.

Aspect 46: The system or application of any of the Aspects disclosed herein, wherein the monitoring device, the medical monitoring device, or the patient monitor is capable of measuring patient EKG, such as a 12-lead EKG or a Holter monitor.

Aspect 47: The system or software or mobile application of any of the Aspects disclosed herein, wherein the control module controls the administering by controlling or instructing the infusion pump concerning one or more of a rate of one or more infusion, a time of infusion, or any interval between one or more infusions.

Aspect 48: The system or application of any of the Aspects disclosed herein, wherein the dosing criteria and/or the IV dosing comprise IV loading dose, any minimum delay to first oral or IV maintenance dose, and/or oral or IV maintenance dosing interval.

Aspect 49: The system or application of any of the Aspects disclosed herein, wherein the antiarrhythmic drug is one or more of sotalol, dofetilide, amiodarone, ibutilide, dronedarone, procainamide, flecainide, or propafenone.

Aspect 50, which is the system of any of the Aspects disclosed herein, wherein the dosing criteria are selected from one or more criteria from labeling instructions for the antiarrhythmic drug, including labeling instructions for any one or more of sotalol, dofetilide, amiodarone, ibutilide, dronedarone, procainamide, flecainide, or propafenone, such as provided in the product labels referenced herein. For example, dosing criteria for any antiarrhythmic drug can follow any of the flowcharts provided in FIG. 1-10 or 13-14, modified according to the label for a particular desired drug being administered.

Aspect 51, which is any of the methods, systems or treatment management applications of Aspects 1-50, adapted for treatment of any one or more of atrial fibrillation (AFL); atrial flutter (AF); ventricular tachycardia; hemodynamically stable or unstable ventricular tachycardia (VT); ventricular fibrillation (VF); paroxysmal supraventricular tachycardia; paroxysmal atrial fibrillation; heart failure; coronary artery disease; pulmonary artery hypertension; maintenance of normal sinus rhythm (delay in time to recurrence of atrial fibrillation/atrial flutter (AFIB/AFL)) for example in patients with symptomatic AFIB/AFL and/or who are currently in sinus rhythm, and/or indicated for the treatment of life-threatening ventricular tachycardia; and/or treating people, such as hospitalized people, with atrial fibrillation cardioverted to normal sinus rhythm, without ventricular arrhythmias or various forms of blocks, in patients with normal kidney function; treating life-threatening recurrent VF or life-threatening recurrent hemodynamically unstable VT; treating paroxysmal or persistent atrial fibrillation or atrial flutter, such as with patients who have experienced a recent AF/AFL episode who are in sinus rhythm or who will be cardioverted; and/or treating Wolff-Parkinson-White syndrome and/or paroxysmal supraventricular tachycardia.

### BRIEF DESCRIPTION OF THE DRAWINGS

The accompanying drawings illustrate certain aspects and principles of the implementations set forth, and should not be construed as limiting.

FIG. 1 is a flowchart depicting a method for determining dosing of an antiarrhythmic drug, such as sotalol hydrochloride, dofetilide, etc., e.g., for implementation by a software app.

FIG. 2 is a flowchart depicting an exemplary method for determining an IV loading dose for initiation of a subject on a target oral dose of 80 mg sotalol hydrochloride.

FIG. 3 is a flowchart depicting an exemplary method for determining an IV loading dose for initiation of a subject on a target oral dose of 120 mg sotalol hydrochloride.

FIG. 4 is a flowchart depicting an exemplary method for determining an IV loading dose for escalation of an 80 mg dose of sotalol hydrochloride up to a target oral dose of 120 mg.

FIG. 5 is a flowchart depicting an exemplary method for determining an IV loading dose for escalation of a 120 mg dose of sotalol hydrochloride up to a target oral dose of 160 mg.

FIG. 6 is a flowchart of an exemplary method for determining an IV loading dose, the minimum delay to a first oral dose and/or a time for maintenance dosing to begin, and the maintenance dosing interval for sotalol hydrochloride.

FIG. 7 is a flowchart of an exemplary method for determining when maintenance dosing for sotalol hydrochloride begins relative to an IV loading dose for sotalol hydrochloride based on renal clearance ability of a subject.

FIG. 8 is a flowchart of an exemplary method for determining a maintenance dosing interval for sotalol hydrochloride.

FIG. 9 is a flowchart depicting a method for monitoring QTc of a subject to determine whether to continue with dosing sotalol hydrochloride or lower the dose.

FIG. 10 is a flowchart depicting a method for monitoring QTc of a subject to determine whether to continue with dosing sotalol hydrochloride or lower the dose, based on a subject's CrCl.

FIG. 11 is an illustration of a pharmacokinetic profile for an intravenous sotalol loading dose followed by subsequent sotalol oral doses in a typical patient.

FIG. 12 is an exemplary system and medical device for determining dosing and optionally administering one or more antiarrhythmic drug, such as sotalol, dofetilide, amiodarone, ibutilide, dronedarone, procainamide, flecainide, and/or propafenone to a subject.

FIG. 13 is a flowchart of an exemplary method for determining a maintenance dosing interval for dofetilide.

FIG. 14 is a flowchart depicting a method for monitoring QTc of a subject to determine whether to continue with dosing dofetilide or lower the dose.

### DETAILED DESCRIPTION

Reference will now be made in detail to various illustrative implementations. It is to be understood that the following discussion of the implementations is not intended to be limiting.

The following figures depict implementations of various methods represented by flowchart diagrams. To the extent the figures or examples herein may refer to administering sotalol in particular, any antiarrhythmic drug, including additionally dofetilide, amiodarone, ibutilide, dronedarone, procainamide, flecainide, and/or propafenone, can be used instead or in combination with sotalol and the amounts of the IV and/or oral dosages can be adjusted appropriately, for example, adjusted according to the labeling instructions for the particular antiarrhythmic being administered instead of or in addition to sotalol. As can be appreciated, the implementations depicted herein can be combined to be performed serially, and/or in parallel. The implementations can be modified from those depicted in the figures to remove or add one or more steps, change the order of steps, or substitute or exchange one or more steps in a manner that achieves the same effective result. It will be understood that each block of the flowchart diagrams, and combinations of blocks in the flowchart diagrams, can be implemented or supported by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart blocks. The method implementations depicted herein can be performed as a series of steps which are implemented as computer-readable code, computer-readable instructions, computer-executable instructions, or “software” performed by one or more processor. Such software can be loaded onto a memory of computer, computing device, medical device, or system thereof, as an application or program capable of performing the steps of the method or otherwise made accessible to such devices through computer networking or cloud computing. Any of the methods can be used with any of the systems described herein and vice versa.

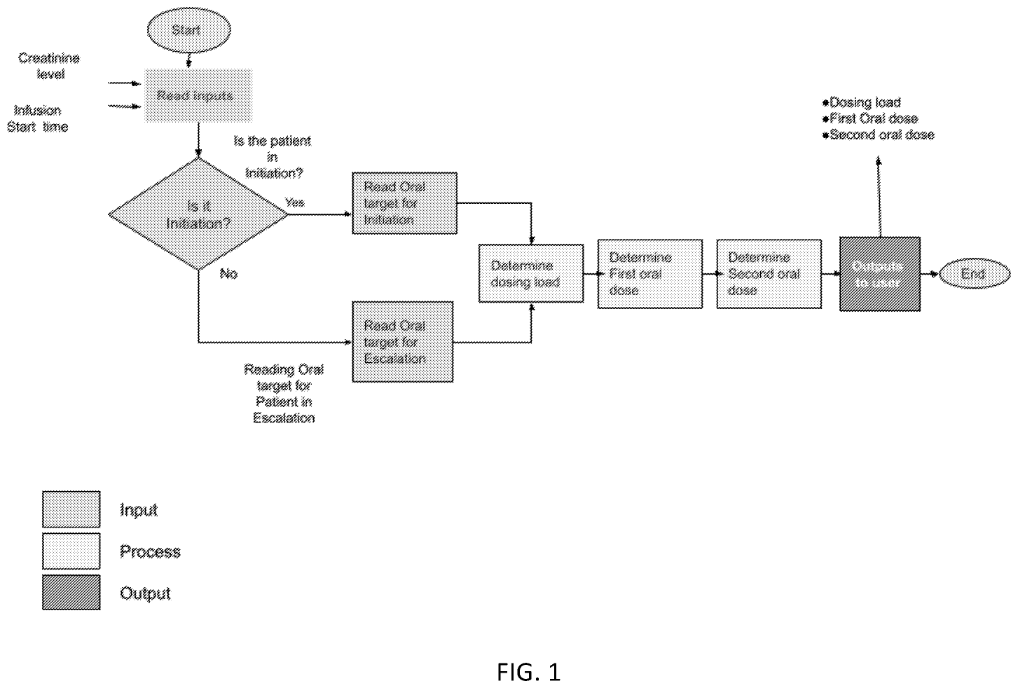

Embodiments of the invention include software for implementing any of the methods described herein, in whole or part. As shown in FIG. 1, the software application can be configured to receive input regarding a subject's creatinine clearance (CrCl) or creatinine level (which can be used to determine a CrCl) and optionally an estimated start time for dosing to begin, such as a start time for sotalol hydrochloride or dofetilide infusion. In any of the figures or embodiments herein, when referring to CrCl, it is also understood that a creatinine level or serum creatinine of a subject/patient can be used as the input instead or in addition to CrCl. The app is additionally configured to receive input regarding whether the subject is being initiated on the drug or whether the subject's dose is being escalated from one oral dose to a higher target oral dose, and the desired oral target dose, such as for sotalol hydrochloride a target oral dose of 80 mg (initiation), 120 mg (initiation or escalation) or 160 mg (escalation). From the inputs, the software application is configured to determine the amount of an IV loading dose expected to initiate or escalate the subject to the desired target oral dose based on the subject's creatinine clearance (which can be determined by the software application using for example the Cockcroft-Gault formulas for creatine clearance from a serum creatinine level of the patient if used as an input). The software application is further configured to determine when a first oral dose should be administered to the subject, optionally relative to the start time of the IV loading dose (e.g., the estimated start time) based on the subject's creatinine clearance, target oral dose and/or whether the subject is being initiated or escalated on the drug. The software application is further configured to determine a maintenance dosing interval for the oral dosing based on the target oral dose, the subject's creatinine clearance and/or whether the subject is being initiated or escalated on the drug. Outputs from the software app can include one or more or all of: (a) a start time for drug infusion to begin and/or end; (b) an amount of drug for administration by way of the IV loading dose or a choice of amounts for the IV infusion, if more than one; (c) an amount of drug for administration by way of oral dosing (e.g., target oral dose); (d) a start time for oral dosing to begin (whether a specific time, such as 9:00 a.m., or an amount of time from completion of the IV dosing, such as at least 4 hours after completion of the infusion); (e) a start time for second and/or subsequent oral doses to begin (whether a specific time or an amount of time following the time of the first oral dose, such as at 12 hour intervals). Optionally, the start time for infusion to begin can be reset and one or more determinations recalculated accordingly. For example, administration of the infusion might actually begin at 9:30 a.m. instead of an expected start time of 9:00 a.m., and the time for the first and/or subsequent oral doses can be redetermined/recalculated (or confirmed, if no change) by the software app accordingly.

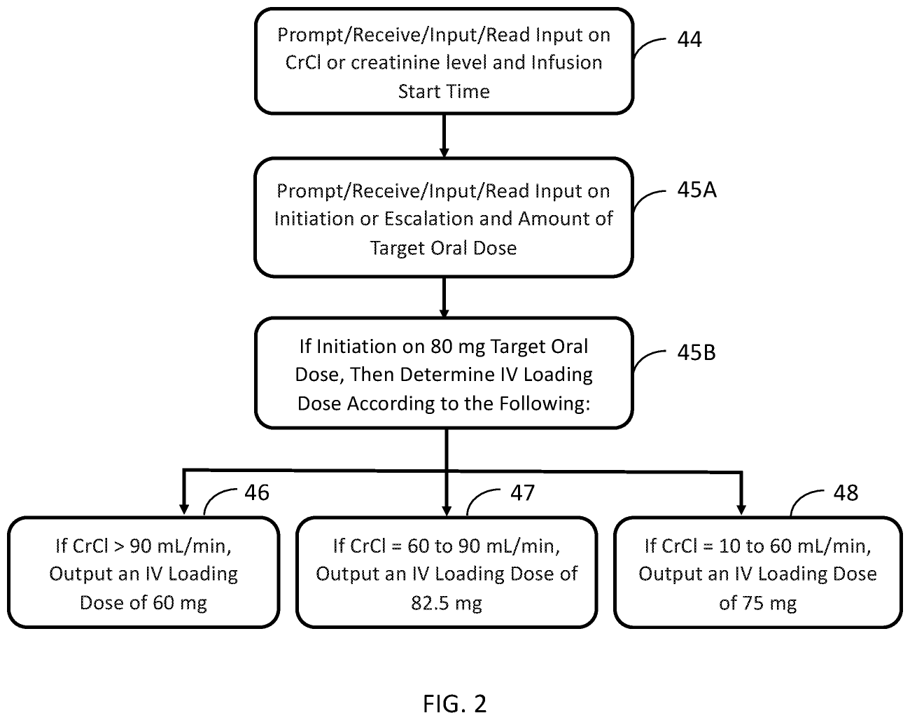

FIGS. 2-10 illustrate specific implementations of methods disclosed herein. The implementations exemplified are not limited to sotalol and can be used to determine dosing and administration of any one or more antiarrhythmic drug, including but not limited to sotalol, dofetilide, amiodarone, ibutilide, dronedarone, procainamide, flecainide, or propafenone.

One implementation of a method of the invention is shown in FIG. 2, which exemplifies a method relating to initiation of a patient on sotalol hydrochloride of a target oral dose of 80 mg. As shown in FIG. 2, a subject's creatinine clearance and start time for infusion can be input or prompted to be input, read and/or received 44. Inputs also include whether the subject is being initiated on or escalated to a higher target oral dose of the drug and the amount of the target oral dose 45A. For initiation of a subject on an 80 mg target oral dose, the amount of the IV loading dose is determined based on the subject's creatinine clearance and is outputted 45B. For sotalol hydrochloride, for example, if the inputted creatinine clearance exceeds 90 mL/min, then an IV loading dose of 60 mg is outputted 46. If the inputted creatinine clearance is within the range of 60 to 90 mL/min, then an IV loading dose of 82.5 mg is outputted 47. If the inputted creatinine clearance is within the range of 10 to 60 mL/min, then an IV loading dose of 75 mg is outputted 48 or a choice is outputted, such as an IV loading dose of 75 mg or 82.5 mg. IV loading doses for other antiarrhythmics is found in the product labels referenced herein and in some cases may be based on either or both of creatinine clearance and/or liver clearance of the patient.

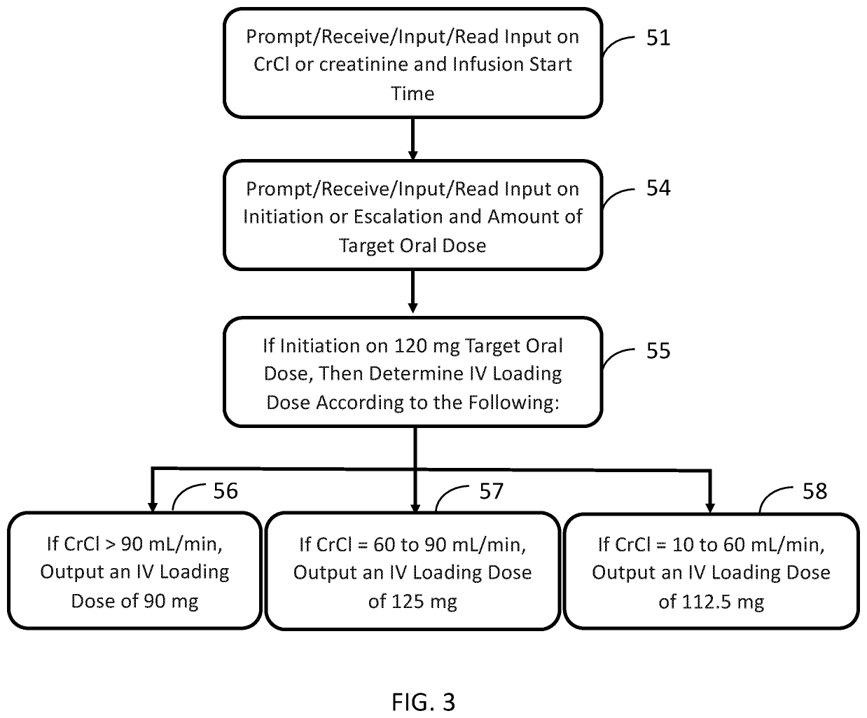

Another implementation of a method of the invention is shown in FIG. 3, which exemplifies a method relating to initiation of a patient on sotalol hydrochloride of a target oral dose of 120 mg. As shown in FIG. 3, a subject's creatinine clearance and start time for infusion can be input or prompted to be input, read and/or received 51. Inputs also include whether the subject is being initiated on or escalated to a higher target oral dose of the drug and the amount of the target oral dose 54. For initiation of a subject on a 120 mg target oral dose, the amount of the IV loading dose is determined based on the subject's creatinine clearance and is outputted 55. For sotalol hydrochloride, for example, if the inputted creatinine clearance exceeds 90 mL/min, then an IV loading dose of 90 mg is outputted 56. If the inputted creatinine clearance is within the range of 60 to 90 mL/min, then an IV loading dose of 125 mg is outputted 57. If the inputted creatinine clearance is within the range of 10 to 60 mL/min, then an IV loading dose of 112.5 mg is outputted 58, or a choice of an IV loading dose of 112.5 mg or 125 mg is outputted.

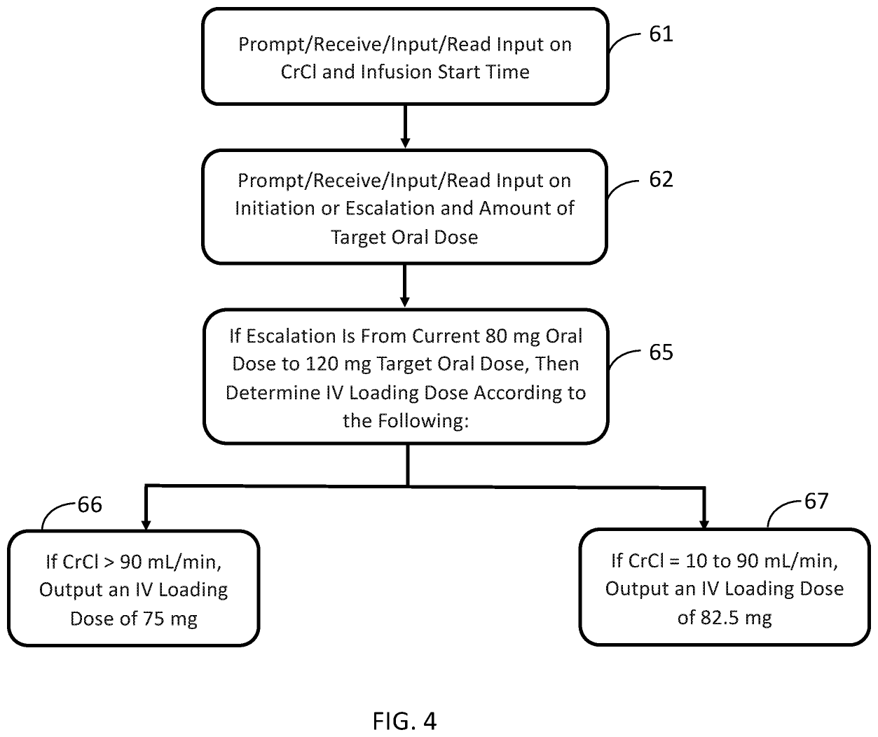

Another implementation of a method of the invention is shown in FIG. 4, which exemplifies a method relating to escalation of a dose of sotalol hydrochloride to a higher target oral dose. As shown in FIG. 4, a subject's creatinine clearance and start time for infusion can be input or prompted to be input, read and/or received 61. Inputs also include whether the subject is being initiated on or escalated to a higher target oral dose of the drug and the amount of the target oral dose 62. For escalation of a subject from a current oral dose of 80 mg to a higher oral dose of 120 mg, the amount of the IV loading dose is determined based on the subject's creatinine clearance and is outputted 65. If the inputted creatinine clearance exceeds 90 mL/min, then an IV loading dose of 75 mg is outputted 66. If the inputted creatinine clearance is within the range of 10 to 90 mL/min, then an IV loading dose of 82.5 mg is outputted 67.

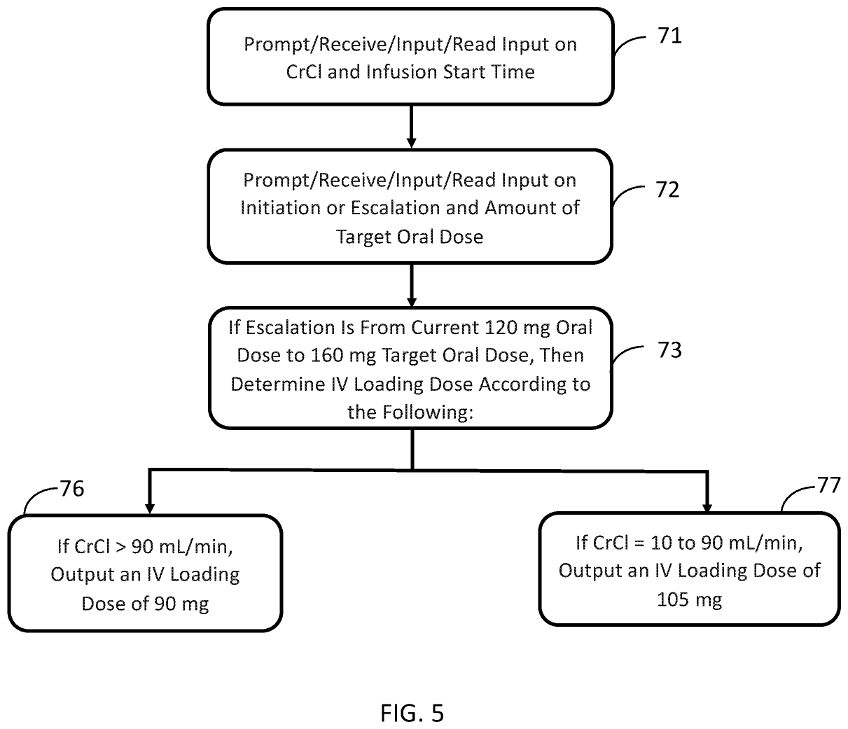

Another implementation of a method of the invention is shown in FIG. 5, which exemplifies a method relating to escalation of a dose of sotalol hydrochloride to a higher target oral dose. As shown in FIG. 5, a subject's creatinine clearance and start time for infusion can be input or prompted to be input, read and/or received 71. Inputs also include whether the subject is being initiated on or escalated to a higher target oral dose of the drug and the amount of the target oral dose 72. For escalation of a subject from a current oral dose of 120 mg to a higher oral dose of 160 mg, the amount of the IV loading dose is determined based on the subject's creatinine clearance and is outputted 73. If the inputted creatinine clearance exceeds 90 mL/min, then an IV loading dose of 90 mg is outputted 76. If the inputted creatinine clearance is within the range of 10 to 90 mL/min, then an IV loading dose of 105 mg is outputted 77.

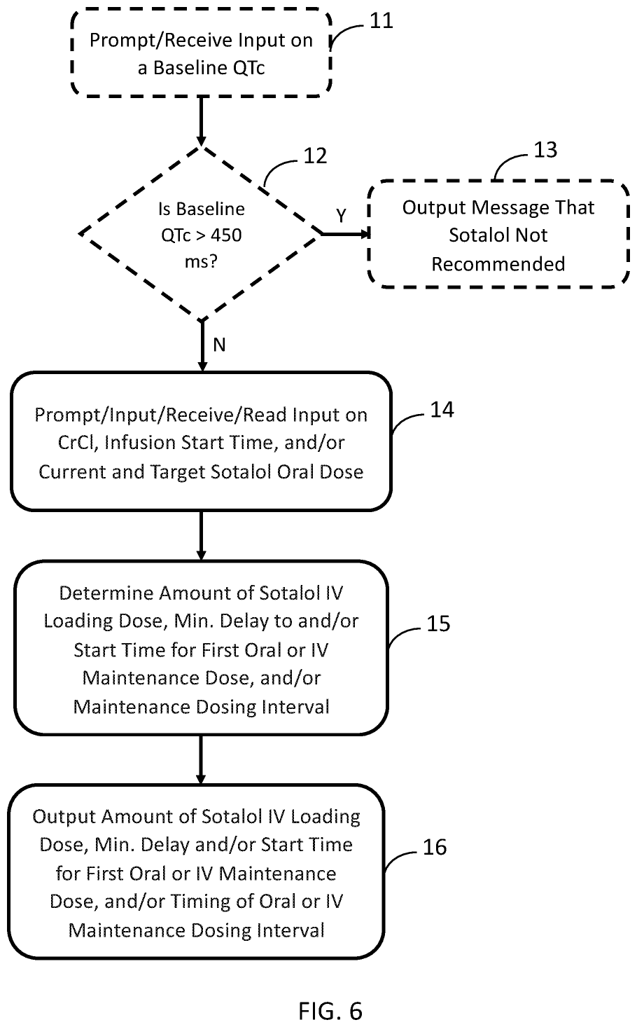

Another implementation of a method for determining dosing and/or administration of an antiarrhythmic drug is shown in FIG. 6. As shown, a subject's creatinine clearance, start time for infusion and/or current and target oral dose amount can be input or prompted to be input, read and/or received 14. Based on the input of the patient's creatinine clearance or liver clearance, current maintenance oral or IV dose, and target maintenance oral or IV dose, an amount of the IV loading dose, the start time of minimum delay (if any) to the first maintenance dose (e.g., oral or IV dose) and/or the oral or IV dosing interval are determined 15. The determined amount for the IV loading dose, the start time for oral dosing or minimum delay (if any) to first oral dose, and/or the maintenance oral or IV dosing interval are then outputted 16.

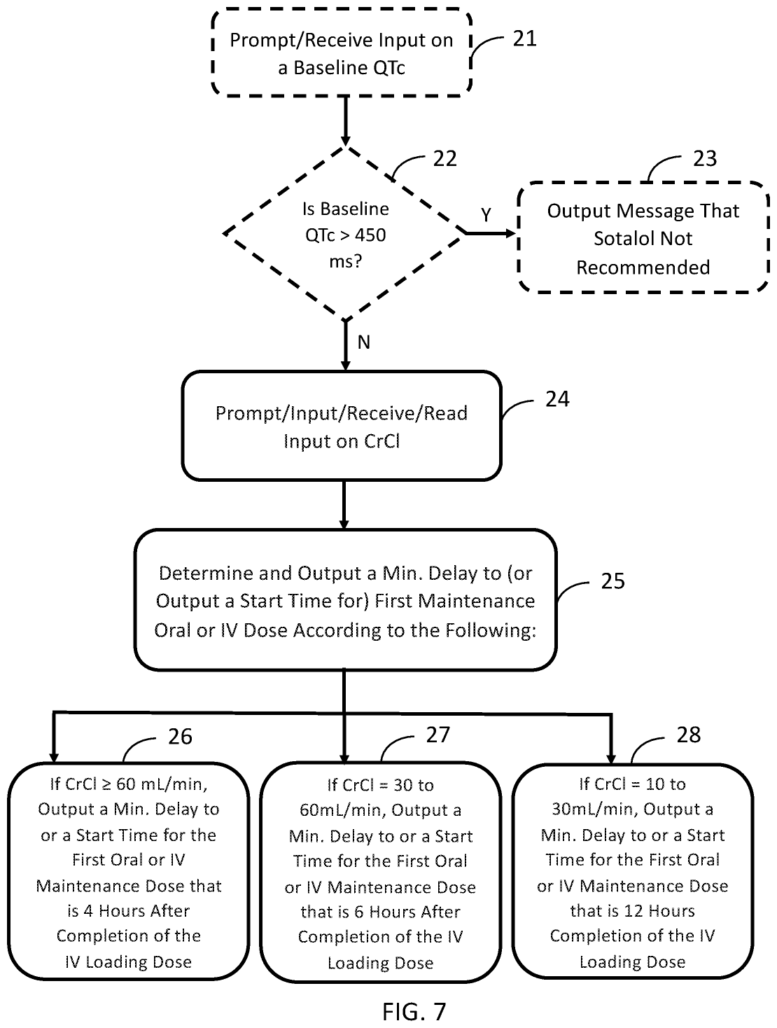

Another implementation of a method is shown in FIG. 7. The method can start with prompting/inputting/receiving/reading an input relating to the patient's kidney and/or liver clearance ability 24 (such as a creatinine clearance). Based on the input of the patient's creatinine clearance and/or liver clearance, the timing of and/or a minimum delay to first oral or IV maintenance dose is determined, and the timing of and/or minimum delay is outputted 25. For sotalol hydrochloride, for example, if the inputted creatinine clearance is 60 mL/min or more, then a minimum delay of 4 hours is outputted 26 (i.e., the practitioner is prompted and/or receives an output that the first oral dose should be administered at least 4 hours or more after completion of the IV, or a start time for the first maintenance dose can be outputted/provided). If the inputted creatinine clearance is within the range of 30 to 60 mL/min, then a minimum delay of 6 hours is outputted 27 (i.e., the first oral dose is instructed to be administered at least 6 hours after completion of the IV and/or a corresponding start time for maintenance dosing can be provided/outputted), or if more than one option applies, a choice of administration times and/or minimum delay to first oral dose can be provided. If the inputted creatinine clearance is within the range of 10 to 30 mL/min, then a minimum of 12 hours is outputted 28 (i.e., the timing of the first oral dose is determined/output as starting at least 12 hours after completion of the IV dose), or a choice of options can be provided. Creatinine clearance and/or liver clearance thresholds for other antiarrhythmics can be found in the labeling information provided in the product labels referenced herein and the corresponding thresholds for those drugs can be used instead if initiating, continuing or administering other antiarrhythmic drugs.

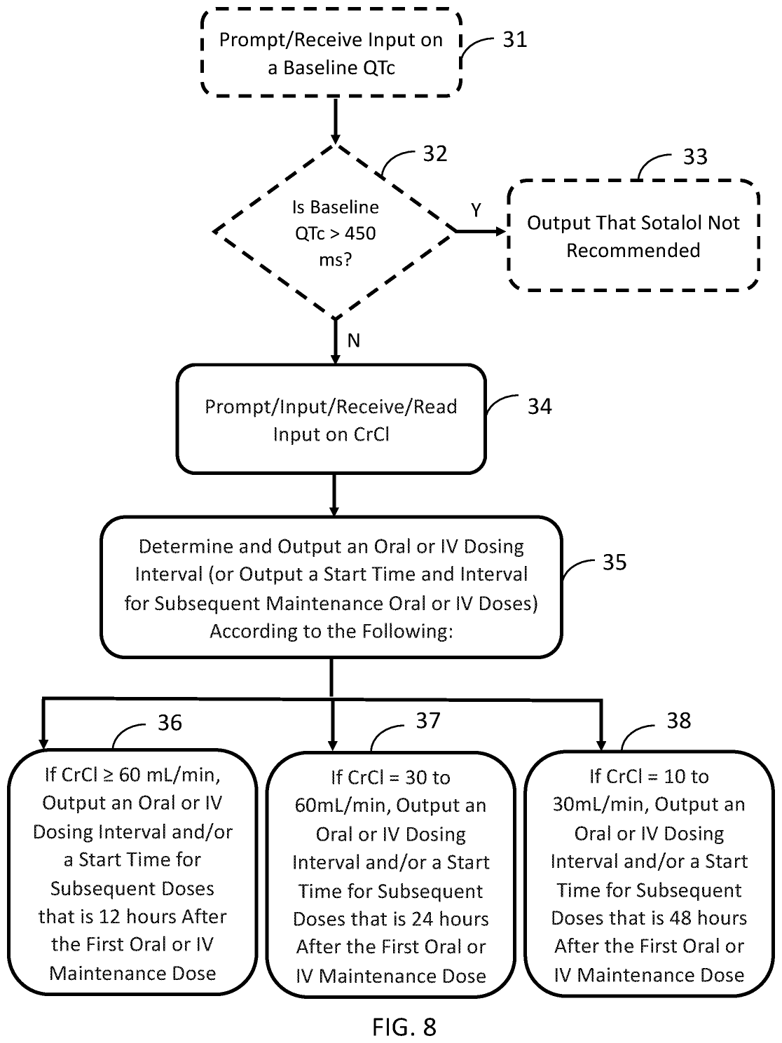

Another implementation of a method is shown in FIG. 8. The method can start with prompting or receiving input of a patient's creatinine clearance 34. For antiarrhythmic drugs where liver clearance is an issue, the health professional can instead or in addition be prompted to enter a value relating to the patient's liver clearance ability or the creatinine clearance can be presented by the user interface by way of a drop down list to be selected. Based on the input of the patient's creatinine clearance or liver clearance capability, an oral or IV dosing interval is determined, and the oral or IV dosing interval is outputted 35. For antiarrhythmic drugs that are administered intravenously, an IV dosing schedule/interval will be outputted (e.g., ibutilide or procainamide). If the inputted creatinine clearance is 60 mL/min or more, then for sotalol hydrochloride a dosing interval of 12 hours is outputted 36 (e.g., administration of subsequent oral and/or IV doses is indicated to be at 12 hour intervals from the first maintenance dose, such as from the first oral dose). For a creatinine clearance input of within the range of 30 to 60 mL/min, then a dosing interval for sotalol of 24 hours is outputted 37 (e.g., administration of subsequent oral and/or IV doses is indicated to be at 24 hour intervals from the first maintenance dose, such as from the first oral dose), or a choice of dosing intervals can be provided if more than one option applies. For a creatinine clearance input of within the range of 10 to 30 mL/min, then a dosing interval for sotalol of 48 hours is outputted 38 (e.g., administration of subsequent oral and/or IV doses is indicated to be at 48 hour intervals from the first maintenance dose, such as from the first oral dose), or a choice of interval options is presented. Maintenance dosing intervals, such as oral dosing intervals, and creatinine clearance or liver clearance thresholds for other antiarrhythmics are provided in the product labels referenced herein and can be substituted in place of those for sotalol if initiating, continuing or administering another antiarrhythmic drug.

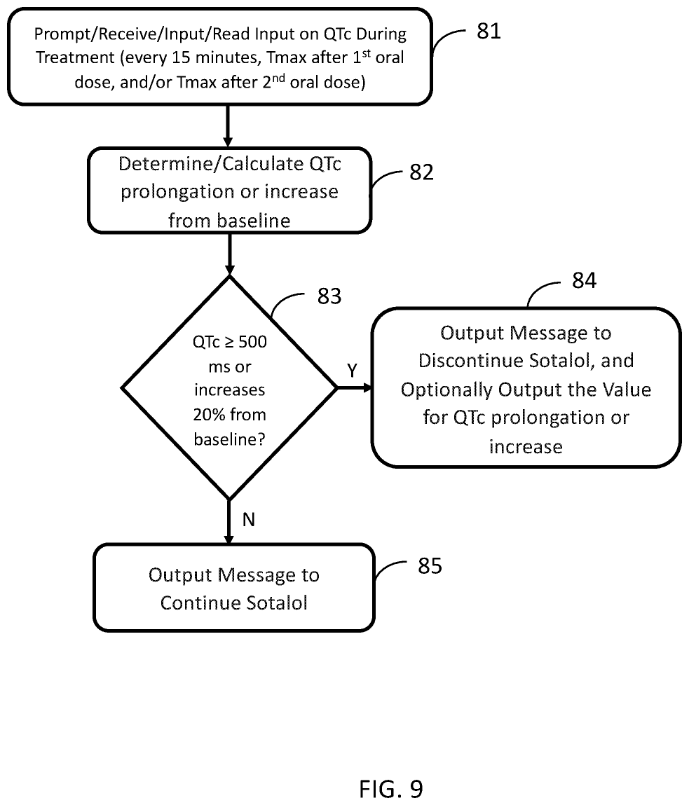

Another implementation of a method is shown in FIG. 9. The method can be implemented for patients having a current sotalol oral dose of 0 mg and a target sotalol oral dose of 80 mg (e.g., initiation), and such values can be inputted along with one or more health characteristics of the patient in steps described for other methods provided herein. Appropriate current and target oral and/or IV doses for other antiarrhythmic drugs can be found in the product labels referenced herein. For sotalol, as an example, a health professional is first prompted to input patient QTc during sotalol treatment 81. The QTc can be measured and then inputted at 15 minute intervals, at or around Tₘₐₓ after a Pt oral dose, and/or at or around Tₘₐₓ after a 2^{nd} oral dose. Then, QTc prolongation or increase from baseline are determined or calculated based on the inputted QTc 82. Then, a determination is made as to whether QTc is equal to or exceeds 500 ms or increases 20% from baseline 83. If QTc is equal to or exceeds 500 ms or increases 20% from baseline, then a message to discontinue sotalol is outputted 84; the QTc value and/or percentage increase from baseline value may also be optionally outputted. If QTc does not equal or exceed 500 ms or increase 20% from baseline, then a message to continue sotalol is outputted 85. Corresponding threshold values for QTc prolongation or increase from baseline can be found in the product labels referenced herein for other antiarrhythmic drugs.

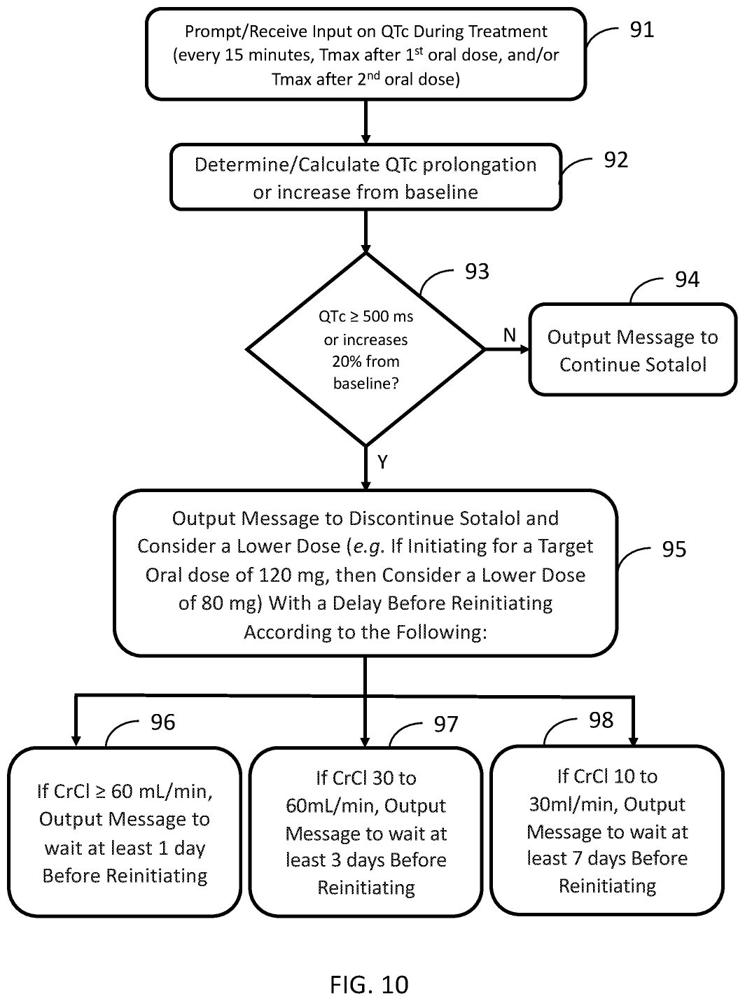

Another implementation of a method is shown in FIG. 10. The method can be implemented for patients having a current sotalol oral dose of 0 mg and a target sotalol oral dose of 120 mg, and such values can be inputted along with one or more health characteristics of the patient in steps described for previous methods. A health professional is first prompted to input a QTc during sotalol treatment 91. The QTc can be measured and then inputted at 15-minute intervals, at or around Tₘₐₓ after a first oral dose, and/or at or around Tₘₐₓ after a second oral dose. Then, QTc prolongation or increase from baseline are determined or calculated based on the inputted QTc 92. Then, a determination is made as to whether QTc equals or exceeds 500 ms or increases 20% from baseline 93. If QTc does not equal or exceed 500 ms or increase 20% from baseline, then a message to continue sotalol is outputted 94. If QTc equals or exceeds 500 ms or increases 20% from baseline, then a message to discontinue sotalol and consider a lower dose (e.g. 80 mg) with a delay before reinitiating is outputted 95; the QTc value and/or percentage increase from baseline value may also be optionally outputted. If the patient's inputted creatinine clearance is greater than or equal to 60 mL/min, then a message is outputted to wait at least 1 day before reinitiating 96. If the patient's creatinine clearance is within the range of greater than or equal to 30 to less than 60 mL/min, then a message is outputted to wait at least 3 days before reinitiating 97. If the patient's creatinine clearance is within the range of ≥10 to <30 mL/min, then a message is outputted to wait at least 7 days before reinitiating 98.

In embodiments, a health care professional can input one or more health characteristics of a patient and one or more oral or IV dosing characteristics of the patient (relating to any one or more of sotalol, dofetilide, amiodarone, ibutilide, dronedarone, procainamide, flecainide, or propafenone). The one or more health characteristics of the patient can include information that can affect one or more pharmacokinetic characteristics or behaviors of sotalol etc. in the patient, such as renal or liver clearance, half-life, bioavailability, absorption, Cₘₐₓ, Tₘₐₓ, and Area under the Curve (AUC). Such health characteristics can include renal function, as indicated by patient creatinine clearance, or liver function, or concomitant medications. The one or more health characteristics can also include information indicating a potential risk of an adverse effect. The adverse effect can include proarrhythmia (e.g. Torsade de Pointes (TdP)), bradycardia, sinus pauses or sinus arrest, hypotension, heart failure, negative inotropy, dyspnea, and/or fatigue, or any other adverse effect available from sotalol pre-marketing or post-marketing studies or reports. Adverse effects for various antiarrhythmics such as sotalol, dofetilide, amiodarone, ibutilide, dronedarone, procainamide, flecainide, or propafenone, can be found in the product labels referenced herein. The information indicating a potential risk can include patient QT interval or QTc or concomitant medications, or any other information relevant to the appropriateness of initiation, escalation or continuation of the antiarrhythmic to be administered. The one or more oral or IV dosing characteristics can include information such as current oral or IV dose of the drug and target oral or IV dose of the drug, such as whether the patient/subject is being initiated on the drug or escalated to a higher oral dose of the drug or switched to another drug. The dosing characteristics can include drug information for an antiarrhythmic drug a patient is currently taking (such as dofetilide) and drug information for a target drug the patient is expected to be switched to (such as sotalol), or vice versa. Then, a determination is made regarding appropriateness of the antiarrhythmic drug for the patient, IV loading dose, or IV or oral dosing protocol characteristics such as minimum delay to first oral or IV maintenance dose, and/or oral or IV dosing interval, based on the input(s). Then, a message is outputted regarding appropriateness of the drug for the patient, IV loading dose, minimum delay to first oral or IV dose, and/or oral or IV maintenance dosing interval, based on the determination(s) made.

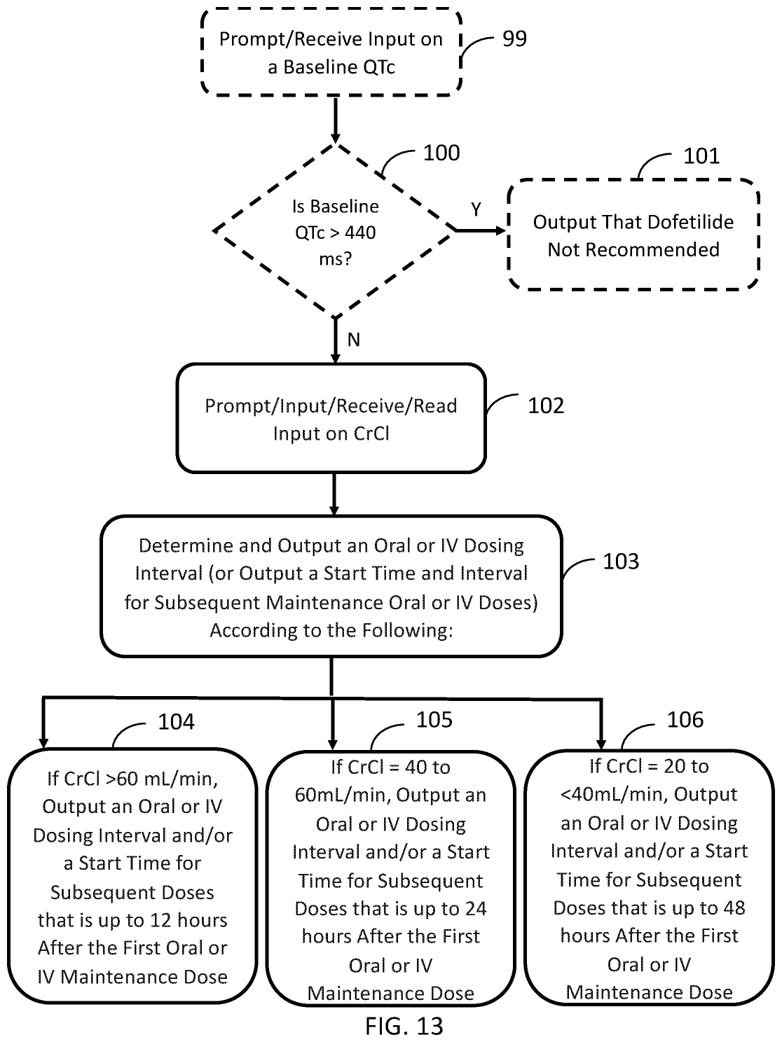

In any embodiment, for example those disclosed in FIGS. 1-10 and 13-14, additional steps can optionally include steps relating to a subject's fitness for a particular antiarrhythmic drug based on QTc measurements. Optionally, a baseline QTc of a patient can be input or prompted to be input, read or received for determining antiarrhythmic drug dosing. For sotalol hydrochloride, a determination can be made as to whether the baseline QTc exceeds a threshold of 450 ms. Other antiarrhythmic drugs may have a different threshold for QTc and can be found in the product labels referenced herein. If the baseline QTc exceeds a threshold of 450 ms, sotalol (or other antiarrhythmic) is not recommended and a corresponding alarm, alert or message can be outputted. If the baseline QTc does not exceed the threshold of 450 ms, additional steps as outlined in any of FIGS. 1-10 can be taken to determine the appropriate dosing for the subject. Exemplary steps relating to measuring QTc are shown by way of the dotted-line portions of FIG. 6 (see 11, 12, 13), FIG. 7 (see 21, 22, 23), FIG. 8 (see 31, 32, 33), and FIG. 13 (see 99, 100, 101). When referring to QTc or a QT interval, either can be used in the embodiments or figures herein and either can be input into the medical device, systems or apps herein and substituted accordingly.

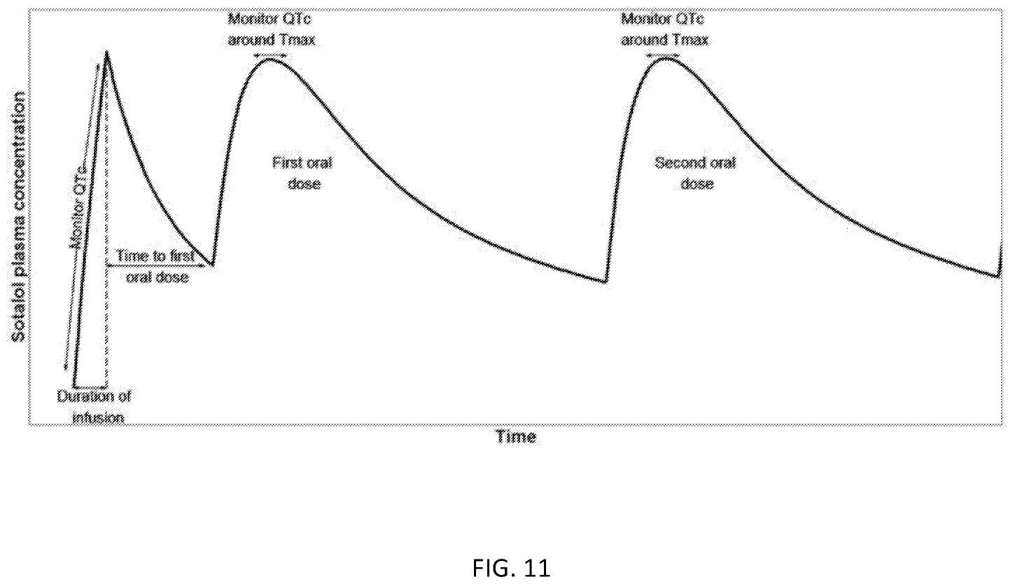

FIG. 11 illustrates implementation of various features of the method implementations in the context of a pharmacokinetic profile of a typical patient. In particular, FIG. 11 illustrates patient QTc monitoring occurs at stages within the pharmacokinetic profile in which the patient is most vulnerable to concentration-dependent adverse effects (e.g. ventricular arrhythmias).

Steps involving measurement of a QTc baseline of a patient or QTc at specified intervals, or measurement of QTc at specific times, are subject to implementation or modification by a qualified physician according to their professional judgment. For example, the QTc baseline can be measured within hours (e.g. 12, 8, 6, 5, 4, 3, 2, 1) or minutes (e.g. 45, 30, 20, 15, 10, 5) prior to initiating a procedure prompted by the method implementations, such as administering a loading dose. The QTc baseline measurements can be taken at multiple times and averaged, or be taken at a single time. Specific intervals for measuring QTc during infusion of the loading dose can include 5, 10, 15, or 20 minutes. Measuring QTc around Tₘₐₓ after a first and/or second oral dose can include measuring QTc at 2 to 4 hours, 1 to 3 hours, or 2.5 to 5 hours after the first and/or second oral dose, and/or adjusting the time of QTc measurement according to the patient's creatinine clearance or other variable potentially affecting sotalol Tₘₐₓ values, such as concomitant medications, and/or adjusting the time of QTc measurement according to sotalol pharmacokinetic data obtained from a clinical population of patients.

Steps involving “prompting”, “inputting”, “calculating”, “determining”, and “outputting” depicted in the preceding figures can be implemented in a variety of ways known in the computer science and engineering arts.

Prompting and inputting steps can be performed through one or more input-output (I/O) interface on the computer or computing device. Inputting can be provided manually from a user and/or provided automatically from any one or more devices or systems for obtaining patient information, such as creatinine clearance, QT interval or QTc. Prompting steps can be performed as visual or auditory cues for the health professional to enter specific patient characteristics. Visual prompting can take the form of a message and/or input field provided to the health professional on a display. The input field can be provided as part of a graphical user interface provided on the display of the computing device which provides one or more data entry fields, check boxes, or pull-downs which allow the health professional to input data. Auditory prompting can take the form of speech or a recording broadcast from a speaker which prompts the health professional to enter the patient characteristics. Inputting steps can be performed through traditional inputting means such as a physical keyboard or a keyboard projected on a display, such as a touchscreen. The keyboard can be a QWERTY keyboard or a condensed keyboard commonly used in electronic devices such as cell phones. Inputting can also be performed through the health professional providing speech which is inputted through a microphone of the computer or computing device and recognized by speech recognition algorithms programmed on the computer or computing device. Inputting can also be performed automatically through another medical device or system that is in operable connection/communication with the inventive device or system, such as an EKG, a database with stored patient data and/or one or more medical monitoring or measuring devices or systems, such as for obtaining a patient creatinine clearance rate.

Calculating and determining steps can be performed by way of a processor coupled with a memory which has patient health characteristic data and sotalol dosing data stored within or accessible to it. Calculating steps can be performed through mathematical operations programmed on the computer or computing device such as multiplication, addition, subtraction, division, logarithmic functions, exponential functions, algorithmic functions, and so on. Determining steps can be performed by retrieving antiarrhythmic drug dosing data stored in a database within the memory of the computer or computing device based on patient characteristics inputted by the health professional or medical monitoring/measuring device(s). For example, the database can store sotalol (or other antiarrhythmic drug) dosing data, such as sotalol/antiarrhythmic IV loading dose, time for administration of or minimum delay (if any) to first oral or IV maintenance dose, and/or oral or IV maintenance dosing interval as arranged in Table 1 (sotalol) below, such as they are stratified according to tiers of creatinine clearance ranges, and/or or sotalol initiation or escalation values. Specific dosing for other antiarrhythmic drugs can be found in the product labels referenced herein and used instead if administering another antiarrhythmic drug. The determining steps can be performed by retrieving sotalol/antiarrhythmic IV loading dose, administration time of or minimum delay (if any) to first oral or IV dose, and/or oral or IV dosing interval stored in the database within the memory of the computer or computing device based on one or more of the patient health characteristics or sotalol/antiarrhythmic dosing characteristics inputted by the health professional or other monitoring/measuring equipment. The retrieving can include matching one or more inputted patient health characteristics or sotalol/antiarrhythmic dosing characteristics with the values or ranges used to stratify sotalol/antiarrhythmic IV loading dose, administration time of or minimum delay (if any) to first oral or IV dose, and/or oral or IV dosing interval values, so that the appropriate sotalol dosing data is retrieved, provided, accepted and/or inputted.

TABLE 1
Recommended Sotalol Loading Dose

| | Intravenous loading dose [mg] to be administered over 1 hour | | | | | Oral |
|---|---|---|---|---|---|---|
| Creatinine | when the oral dose is going from . . . | | | | Minimum delay to | dosing |
| Clearance* | Sotalol Initiation | | Sotalol Escalation | | first oral dose | interval |
| [mL/min] | 0 to 80 mg** | 0 to 120 mg | 80 to 120 mg | 120 to 160 mg | [hours] | [hours] |
| >90 | 60 | 90 | 75 | 90 | 4 | 12 |
| 60-90 | 82.5 | 125 | 82.5 | 105 | 4 | 12 |
| 30-60 | 75 | 112.5 | 82.5 | 105 | 6 | 24 |
| 10-30 | 75 | 112.5 | 82.5 | 105 | 12 | 48 |

*Calculated using Cockeroft Gault formula
**Recommended starting dose

The sotalol/antiarrhythmic dosing data (e.g. intravenous loading doses, minimum delay to first oral dose, and oral dosing interval) in Table 1 (e.g., sotalol) can be determined from computer-based simulations incorporating sotalol dose-exposure-QTc relationships, and can be modified according to additional computer-based simulations. The computer-based simulations can be based upon mathematical functions, equations, or algorithms which represent the sotalol dose-exposure-QTc relationships, which can be based on theoretical considerations and/or determined empirically.

Outputting steps can be formed through a visual output device such as a display on the computer or computing device, an auditory output device such as a speaker, and/or through other output such as one or more lights (e.g. LEDs). The output can be in the form of text, images and/or video providing instructions on the display to the health professional, and/or through audio messages broadcast on the computer or computing device's audio output device providing such instructions.

The computer-executable code or instructions can be implemented as software or an application capable of being run on a computer or computing device such as a desktop computer or a portable computer/computing device, such as a tablet, laptop, PDA, or smartphone. The computer-executable code or instructions can be installed on the computer or computing device during manufacture as software, or implemented as firmware or hardware (e.g. circuitry), or some combination of these. The computer-executable code or instructions can be configured to be downloadable onto the memory of the computer or computing device from a cloud storage source available on the internet, such as an application retail source (e.g. “app store”) capable of being accessed from a mobile phone, tablet, desktop computer, or other programmable device having components or features capable of performing the method steps described above. Examples of suitable internet-accessible sources include the Apple Store, Google Play, and other sites that make software applications and other downloads available for purchase or license.

The computer-readable code, computer-readable instructions, computer-executable instructions, or “software” can be organized into routines, subroutines, procedures, objects, methods, functions, or any other organization of computer-executable instructions that is known or becomes known to a skilled artisan in light of this disclosure, and can be programmed in any suitable programming language, including JavaScript, C, C #, C++, Java, Python, Perl, Ruby, Swift, Visual Basic, and Objective C. By such programming, the computer-readable code, computer-readable instructions, computer-executable instructions, or “software” instruct a processor of the computer or computing device to carry out the operations and commands of the application. Inputted patient health characteristic data, sotalol dosing data, and computer-executable instructions can be stored in the computer or computing device's memory. The memory can be implemented through non-transitory computer-readable storage media such as RAM. As used in the context of this specification, a “non-transitory computer-readable storage medium (or media)” may include any kind of computer memory, including magnetic storage media, optical storage media, nonvolatile memory storage media, and volatile memory. Non-limiting examples of non-transitory computer-readable storage media include floppy disks, magnetic tape, conventional hard disks, CD-ROM, DVD-ROM, BLU-RAY, Flash ROM, memory cards, optical drives, solid state drives, flash drives, erasable programmable read only memory (EPROM), electrically erasable programmable read-only memory (EEPROM), non-volatile ROM, and RAM.

Additional implementations include a computer, computing device, medical device, or system capable of carrying out the method implementations. The computer, computing device, medical device, or system can include one or more processors capable of executing the computer-readable code, computer-readable instructions, computer-executable instructions, or “software”, one or more interface capable of providing input or output, one or more databases and a set of instructions (e.g. software) stored in a memory of the computer, computing device, medical device, or system for carrying out the method implementations. The computer, computing device, medical device or system can include a stand-alone computer, such as a desktop computer, a portable computer, such as a tablet, laptop, PDA, watch, or smartphone, or a set of computers or devices connected through a network including a client-server configuration and one or more database servers. The network can use any suitable network protocol, including IP, UDP, or ICMP, and may be any suitable wired or wireless network including any local area network, wide area network, Internet network, telecommunications network, Wi-Fi enabled network, or Bluetooth enabled network. In this way, one or more steps of the method implementations can be distributed across the system, or performed directly on a smartphone, computer, computing device, or medical device, according to specific applications or designs.

In another implementation, the system includes 1) one or more computer that is located off-site from a health care environment and 2) one or more computing device located within a health care environment. The off-site computer can be a fixed position computer, and the computing device within the health care environment can be of the desktop variety, or mobile, portable, handheld or worn by a user. The off-site computer can be connected to the internet and can have an internal database capable of housing data, such as patient health characteristic data inputted from the mobile or portable computing device as well as sotalol dosing data, which database is stored in the memory of the off-site computer. The database can be a standard relational database that communicates by way of a database server with the mobile computing device. The off-site computer can include a set of computer-executable instructions stored in memory which can be used to perform the calculating and determining steps off-site. The off-site computer can then communicate to the portable computing device or smartphone the results of such calculating and determining steps as well as messages to be outputted on the portable computing device.

Embodiments include a medical device, such as an electronic device (e.g., handheld electronic device), and/or a medical device comprising software with a graphical user interface and/or software that is accessed through hardware that provides for user interface through screen display(s). In embodiments, the medical device is a software based calculator that uses an algorithm to determine/calculate a loading dose of an antiarrhythmic drug (such as from a table), such as sotalol hydrochloride IV (e.g., using Table 1), using inputted/retrieved data, for example inputted data relating to a creatinine clearance of a subject (which can be inputted manually by a user and/or inputted automatically from another medical device/system), and current and/or target use of the drug for the subject (such as a whether the subject is being initiated on or escalated to a higher oral dose of the drug) and/or the target oral maintenance amount of the drug. In embodiments, the medical device is a quick response code (QR code) contained in a product label, such as labeling for sotalol hydrochloride IV (or any of the product labels referenced herein), which QR code provides digital access (e.g., by scanning the QR code with a mobile device/phone) to a dosing calculator for healthcare professionals, which is intended to recommend dosing parameters for administering IV and/or oral sotalol hydrochloride to a patient. Such medical devices can comprise software stored on an electronic device configured to (i) read, prompt input of and/or accept as inputs: a projected start time for sotalol hydrochloride infusion; creatinine clearance (CrCl) of a subject; whether the subject is being initiated on or escalated to a higher oral dose of the sotalol hydrochloride; and an amount of an oral target dosage of sotalol hydrochloride for the subject; and (ii) execute computer executable instructions to derive from a set of rules (e.g., rules based on criteria set forth in Table 1) a sotalol hydrochloride dosing protocol for the subject based on one or more of the inputs, the dosing protocol comprising: at least an amount of the sotalol hydrochloride for infusion as a loading dose; and optionally a time for a first oral dose of sotalol hydrochloride and/or a time for a second oral dose of sotalol hydrochloride, or oral dosing interval; and (iii) output the sotalol hydrochloride dosing protocol, for example to a healthcare professional.

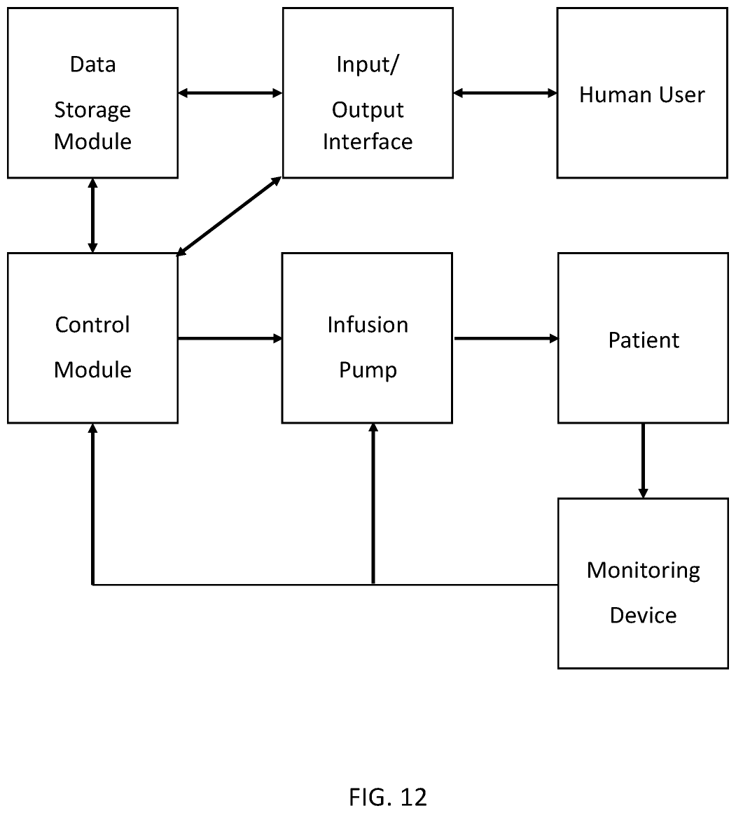

An exemplary system is provided in FIG. 12, which illustrates a medical device and system for determining dosing and optionally administering one or more antiarrhythmic drug, such as sotalol (e.g., sotalol hydrochloride IV), dofetilide, amiodarone, ibutilide, dronedarone, procainamide, flecainide, and/or propafenone to a subject. In embodiments, the system can comprise a dosing calculator comprising a standalone medical device, such as a handheld electronic device, comprising i) an input/output graphical user interface; ii) a memory configured to store dosing criteria for sotalol hydrochloride (e.g., Table 1), store data relating to a subject's current and/or target antiarrhythmic drug dosing amounts, such as sotalol hydrochloride dosing amounts, and store computer-executable instructions for analyzing the subject's dosing related data according to the dosing criteria; iii) a computer processor operatively connected to the memory configured to analyze the data relating to the patient and to identify one or more acceptable IV dose of an antiarrhythmic drug, such as sotalol hydrochloride. Such dosing and administering can be employed for treating one or more cardiovascular condition of a patient, such as atrial fibrillation, atrial flutter, ventricular tachycardia, hemodynamically stable or unstable ventricular tachycardia, ventricular fibrillation, paroxysmal supraventricular tachycardia, paroxysmal atrial fibrillation, heart failure, coronary artery disease, or pulmonary artery hypertension; maintenance of normal sinus rhythm (delay in time to recurrence of atrial fibrillation or atrial flutter (AFIB/AFL)) for example in patients with symptomatic AFIB/AFL and/or who are currently in sinus rhythm, and/or indicated for the treatment of life-threatening ventricular tachycardia; and/or treating people, such as hospitalized people, with atrial fibrillation cardioverted to normal sinus rhythm, without ventricular arrhythmias or various forms of blocks, in patients with normal kidney function; treating life-threatening recurrent VF or life-threatening recurrent hemodynamically unstable VT; treating paroxysmal or persistent atrial fibrillation or atrial flutter, such as with patients who have experienced a recent AFIB/AFL episode who are in sinus rhythm or who will be cardioverted; and/or treating Wolff-Parkinson-White syndrome and/or paroxysmal supraventricular tachycardia. In embodiments, the dosing protocol, such as the IV dose of sotalol hydrochloride, can be input into a control module from the dosing calculator, optionally operably connected with the medical device dosing calculator, for controlling an infusion pump, also optionally operably connected with the control module, according to the dosing protocol to administer the IV sotalol to a patient. Feedback can be provided by a monitoring device, such as an EKG, and input into the control module for adjusting the administering at any point depending on how the subject is responding to the IV treatment, such as IV sotalol treatment.

In implementations, one or more steps of the method implementations can be performed on another medical device, such as an EKG or other medical measuring device. The EKG measuring device can be a 12-lead EKG, or a Holter monitor. The EKG measuring device can include any computer component necessary for performing one or more steps of the method implementations, including one or more processor, a memory, and any component capable of providing input or output. The EKG measuring device can include any computer-readable code, computer-readable instructions, computer-executable instructions, or “software” for performing one or more steps of the method implementations described or depicted herein. In some implementations, the EKG measuring device bypasses the prompting/receiving QTc input steps of the method implementations, such as steps 11, 21, 31, 41, 51, 61, 71, 81, 91, so that these steps are not performed, and instead provides the QTc values or determinations directly for subsequent steps, such as determining whether QTc exceeds a threshold (e.g. 450 ms), such as steps 12, 22, etc. and/or increases from baseline QTc over a percentage threshold, such as steps 83 and 93. The determinations performed on the EKG measuring device can include operations such as comparison to a stored QTc value, multiplication, addition, subtraction, division, logarithmic functions, exponential functions, algorithmic functions, and so on. In some implementations, one or more additional steps of the method implementations are performed on the EKG measuring device, such as those involving outputting a message to continue or discontinue sotalol (e.g. steps 84, 84, 94, 95), or a message to recommend or not recommend sotalol (e.g. steps 13, 23, 33, and so on). The EKG device can also be equipped with a wired (e.g. USB) or wireless (e.g. Wi-Fi, BLUETOOTH®) connection capable of communicating information or data (e.g. QTc values or determinations) with a computer or computing device that is equipped to perform subsequent steps of the method implementation based on the communicated information or data. In some implementations, all of the steps of one or more of the method implementations depicted in FIGS. 1-10 are performed on the EKG measuring device.

Computers, computing devices, or medical devices described herein can include a variety of components known in the art, including one or more processor, a volatile memory, a non-volatile memory, standard I/O interfaces such as a universal serial bus (USB) port, an HDMI or HDMI ARC port, an optical port, an ethernet port, and/or a serial port, a hard drive, a disk drive, a CD-ROM drive, a motherboard, a printed circuit board (PCB), circuitry for enabling a wireless connection or communication to another device, such as a BLUETOOTH® board, a WiFi board, or a transmitter-receiver for mobile telecommunications, a data bus, an address bus, a control bus, and/or one or more user interface devices including a display, keyboard, keypad, trackpad, mouse, control panel, touch screen display, speaker, camera, and/or microphone. The computers, computing devices, or medical devices can be equipped with an operating system implemented as software or firmware. As can be appreciated, the computers, computing devices, or medical devices may differ in their inclusion, exclusion, or configuration of components according to their individual applications or designs.

The sotalol/antiarrhythmic dosing methods, devices, and systems described herein can be used as follows. A patient presents to a health care facility with a condition suitable for treatment with sotalol, such as symptomatic AFIB/AFL. A health care professional, such as a physician, determines that the patient may be a candidate for sotalol/antiarrhythmic treatment based on a clinical evaluation of the patient's symptoms, consideration of possible contraindications, and other patient health information. The health care professional uses a computing device, such as a smartphone or tablet, loaded with a software application capable of performing one or more of the methods described herein, to determine sotalol/antiarrhythmic suitability, to determine suitable choices of antiarrhythmic for the particular patient, and/or to determine an appropriate IV loading dose, and/or determine an appropriate oral or IV dosing protocol, by following the prompting of the application and entering appropriate information. In embodiments, some or all of the health characteristics or patient measurements (such as vitals) can be entered automatically from other medical devices monitoring or assessing the patient. The output of the application informs the health care professional of such suitability, appropriate IV loading dose, and/or appropriate oral or IV maintenance dosing protocol.

Implementations and features of the sotalol/antiarrhythmic dosing methods, devices, and systems are provided below, as well as guidelines and procedures informing their use or application. All values provided in Table 1 and FIGS. 1-10 are subject to modification according to the following.

AFIB is Atrial Fibrillation.

AFL is atrial flutter.

AFIB/AFL=atrial fibrillation and/or atrial flutter.

IV is intravenous.

PO means “per os” and refers to an oral dosing regimen.

BID means “bis in die” and means twice a day.

QD means “quaque die” and means once a day.

QID means “quater in die” and means four times a day.

Patient (or subject) refers to a human patient.

BP is blood pressure.

HR is heart rate.

Renally impaired refers to patients having creatine clearance rates of <60 mL/min.

Sotalol and sotalol hydrochloride (used interchangeably herein) refer to d,1-sotalol hydrochloride which has been approved by the FDA for intravenous administration over 5 hours or oral administration (e.g., 80 mg, 120 mg, and 160 mg tablets).

In an example, sotalol injection is supplied in 10 mL single-dose vials, each containing 150 mg of sotalol hydrochloride as a clear solution (15 mg/mL).

Sotalol Hydrochloride is Contraindicated in Patients with:

Sinus bradycardia (<50 bpm), sick sinus syndrome or second or third degree AV block without a pacemaker

Congenital or acquired long QT syndromes, QT interval >450 ms

Cardiogenic shock, decompensated heart failure

Serum potassium <4 mEq/L

Bronchial asthma or related bronchospastic conditions

Known hypersensitivity to sotalol

Sotalol Indications:

Delay in Recurrence of Atrial Fibrillation or Atrial Flutter:

Sotalol is indicated for the maintenance of normal sinus rhythm (delay in time to recurrence of atrial fibrillation or atrial flutter (AFIB/AFL)) in patients with symptomatic AFIB/AFL who are currently in sinus rhythm.

Life-Threatening Ventricular Arrhythmia:

Sotalol is also indicated for the treatment of life-threatening ventricular tachycardia.

For either indication, intravenous sotalol, when used as a loading dose, achieves steady state concentration faster compared to the conventional oral dosing (e.g., typically 3-days for a non-renally impaired patient).

Typically, IV sotalol is diluted for infusion. For example, IV sotalol can be diluted in saline, 5% dextrose in water (D5 W), or Ringer's lactate. The dilution volume chosen is one that is convenient for administration and consistent with fluid restriction. A volumetric infusion pump can be used to administer the IV sotalol.

Typically, other antiarrhythmic therapy is withdrawn prior to starting sotalol, but in some cases may be administered simultaneously with sotalol, or combinations of other antiarrhythmics can also be contemplated. In some cases, switching from one antiarrhythmic drug to another may also be contemplated, for example, switching a patient from dronedarone therapy to amiodarone therapy or vice versa, or from ibutilide to sotalol or vice versa.

In embodiments, a system for controlling administration of one or more antiarrhythmic drug to a patient is provided comprising:
i) an input/output interface configured to accept and display patient data obtained from user input and/or one or more monitoring device configured to monitor one or more physiological measurement of a patient, such as creatinine clearance, QT interval or QTc;
ii) a data storage module configured to store the patient data, the one or more physiological measurement of the patient and one or more dosing criteria for administering one or more antiarrhythmic drug to the patient; and
iii) one or more control module comprising one or more processor for executing computer-readable instructions for:
a) determining an intravenous dose of the antiarrhythmic drug based on one or more of the patient data, one or more of the physiological measurement of the patient, and the dosing criteria; and
b) providing instructions for controlling and/or controlling an infusion pump to administer the intravenous dose to the patient.

In embodiments, the dosing criteria for the software applications and systems described herein can be provided from labeling instructions for any antiarrhythmic drug, including but not limited to those provided in the product labels referenced herein for sotalol, dofetilide, amiodarone, ibutilide, dronedarone, procainamide, flecainide, or propafenone.

Dosing Criteria:

In embodiments, the IV loading dose for the dosing criteria depends on the target oral or IV dose and creatinine clearance of the patient indicated for oral sotalol. The dosing interval for oral or IV administration of sotalol and/or the administration time or the minimum delay (if any) between the end of the loading infusion and the first oral or IV dose also depend on renal function. For other antiarrhythmics creatinine clearance and/or liver clearance may need to be accounted for when determining the IV loading dose. Although specific examples are provided herein relating to sotalol, depending on the particular antiarrhythmic selected (including additionally dofetilide, amiodarone, ibutilide, dronedarone, procainamide, flecainide, or propafenone, or any antiarrhythmic drug), the dosing can be adjusted up or down to be commensurate with the pharmacokinetics of the drug being administered and reference can be made to the product labels referenced herein for criteria for other specific antiarrhythmic drugs.

Thus, in an example, one implementation provides a software application for performing a method of initiating or escalating sotalol therapy, comprising: administering to a patient in need thereof (a patient for whom sotalol is indicated) an IV and oral sotalol dosage (or other antiarrhythmic drug) based on the patient's creatine clearance (CrCl mL/min), such as wherein the dosages and timing are selected from Table 1.

The Cockcroft-Gault Formulas for Creatine Clearance (CrCl) are:

CrCl (male)=((140−age)×weight in kg)/(serum creatinine×72)

CrCl (female)=CrCl (male)×0.85

Recommended starting dose (80 mg) is the FDA recommended dosage. A physician can select to start a patient on a higher dosage (e.g., 120 mg), if deemed appropriate.

Minimum delay to first oral or IV dose is the time from the end of the loading IV infusion to the first oral or IV dose, which can be referred to as an oral or IV maintenance dose.

Oral or IV dosing interval refers to the time between oral or IV dosages. 12 h is B.I.D. (or BID). 24 h is Q.D. (or QD). 48 hr is according to a 2-day or every other day interval. Appropriate oral or IV dosing intervals for other antiarrhythmics can be found in the product labels referenced herein and can be substituted accordingly.

The IV loading dose for the dosing criteria, as shown in Table 1 (e.g., sotalol), is typically administered (infused) over 1 hour. Additional examples include 50-70 minutes. Further examples include 50, 51, 52, 53, 54, 55, 56, 57, 58, 59, 60, 61, 62, 63, 64, 65, 66, 67, 68, 69, and 70 minutes. For other antiarrhythmic drugs (e.g., dofetilide, amiodarone, ibutilide, dronedarone, procainamide, flecainide, or propafenone, or any antiarrhythmic drug), the duration of the IV administration can be adjusted up or down to be commensurate with the pharmacokinetics of the particular drug being administered. For example, infusion rates for the loading dose for other antiarrhythmics can be found in the product labels referenced herein.

Table 1 shows that the IV loading dose for initiation of a target dose of 80 mg are 60 mg (>90 mL/min CrCl), 82.5 mg (60-90 mL/min CrCl), and 75 mg (30-60 mL/min CrCl and 10-30 mL/min CrCl). Additional examples of the IV loading dose for a target oral dose of 80 mg include in the range of 55-85 mg. Further examples include 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, and 85 mg.

Table 1 shows that the IV loading dose for initiation of a target dose of 120 mg are 90 mg (>90 mL/min CrCl), 125 mg (60-90 mL/min CrCl), and 112.5 mg (30-60 mL/min CrCl and 10-30 mL/min CrCl). Other examples of the IV loading dose for a target dose of 120 mg include 75-135 mg. Further examples include 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, and 135 mg.

Table 1 shows that the IV loading dose for escalation from 80 mg to 120 mg are 75 mg (for >90 mL/min CrCl), and 82.5 (for 60-90 mL/min CrCl, 30-60 mL/min CrCl, and 10-30 mL/min CrCl). Additional examples of the IV loading dose for escalation to 120 mg include 65-90 mg. Further examples include 65, 66, 67, 68, 69, 70, 71, 72, 73, 74, 75, 76, 77, 78, 79, 80, 81, 82, 83, 84, 85, 86, 87, 88, 89, and 90 mg.

Table 1 shows that the IV loading dose for escalation from 120 to 160 mg are 90 mg (>90 CrCl), and 105 (60-90 CrCl, 30-60 CrCl, and 10-30 CrCl). Additional examples of the IV loading dose for escalation to 160 mg include 80-120 mg. Further examples include 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, and 120 mg.

As can be seen in Table 1 (for sotalol), the time of when oral dosing (or IV dosing, if applicable) begins depends on the CrCl of the patient. With respect to some drugs for which liver clearance is alternatively or in addition a factor to consider, threshold liver clearance values can be provided by the dosing criteria of the system. Oral dosing for CrCl of >90 mL/min and 60-90 mL/min typically begins 4 h after IV infusion (e.g., 5 hours after the start of a 1 h IV). Oral dosing for CrCl of 30-60 mL/min typically begins 6 h after IV infusion (e.g., 7 h after the start of a 1 h IV). Oral dosing for CrCl of 10-30 mL/min typically begins 12 h after IV infusion (e.g., 13 h after the start of a 1 h IV). Such minimum delays can also be applicable to starting on an IV maintenance dose after the IV loading dose. For other antiarrhythmic drugs, depending on the particular antiarrhythmic drug (e.g., dofetilide, amiodarone, ibutilide, dronedarone, procainamide, flecainide, or propafenone) to be administered, the timing of administration of the oral or IV dose relative to the IV loading dose can be adjusted up or down to be commensurate with the pharmacokinetics of the drug being administered.

Additional examples of when the oral or IV dosing begins for a CrCl of >90 include 2-6 h after completion of infusion. Further examples include 2, 3, 4, 5, to 6 h or longer such as at least 2 hours, at least 3 hours, at least 4 hours, at least 5 hours, at least 6 hours, at least 7 hours, at least 8 hours, at least 9 hours, at least 10 hours, at least 11 hours or at least 12 hours.

Additional examples of when the oral or IV dosing begins for a CrCl of 60-90 include 2-6 h after completion of infusion. Further examples include 2, 3, 4, 5, to 6 h or longer such as at least 2 hours, at least 3 hours, at least 4 hours, at least 5 hours, at least 6 hours, at least 7 hours, at least 8 hours, at least 9 hours, at least 10 hours, at least 11 hours or at least 12 hours.

Additional examples of when the oral or IV dosing begins for a CrCl of 30-60 include 4-8 h after completion of infusion. Further examples include 4, 5, 6, 7, to 8 h or longer such as at least 4 hours, at least 5 hours, at least 6 hours, at least 7 hours, at least 8 hours, at least 9 hours, at least 10 hours, at least 11 hours or at least 12 hours.

Additional examples of when the oral or IV dosing begins for a CrCl of 10-30 include 10-14 h after completion of infusion. Further examples include 10, 11, 12, 13, to 14 h or longer such as at least 10 hours, at least 11 hours, at least 12 hours, at least 13 hours or at least 14 hours.

As can be seen in Table 1, the oral dosing interval depends on the CrCl of the patient. Oral dosing for CrCl of >90 and 60-90 is typically at 12 h intervals (BID). Oral dosing for CrCl of 30-60 is typically at a 24 H interval (QD). Oral dosing for CrCl of 10-30 is typically at a 48 h interval. For other antiarrhythmic drugs, depending on the particular antiarrhythmic drug (e.g., dofetilide, amiodarone, ibutilide, dronedarone, procainamide, flecainide, or propafenone) to be administered, the dosing interval for subsequent oral or IV doses can be adjusted up or down to be commensurate with the pharmacokinetics of the drug being administered.

Also, Table 1 shows that the amount of sotalol hydrochloride appropriate for a subject with a CrCl of 10-30 mL/min or 30-60 mL/min is, surprisingly, higher than is appropriate for a subject with a CrCl of >90 mL/min.

Additional examples of the oral dosing interval for a CrCl of >90 include 8-16 h after completion of infusion. Further examples include 8, 9, 10, 11, 12, 13, 14, 15, to 16 h.

Additional examples of the oral dosing interval for a CrCl of 60-90 include 8-16 h after completion of infusion. Further examples include 8, 9, 10, 11, 12, 13, 14, 15, to 16 h.

Additional examples of the oral dosing interval for a CrCl of 30-60 include 20-28 h after completion of infusion. Further examples include 20, 21, 22, 23, 24, 25, 26, 27, to 28 h.

Additional examples of when the oral dosing begins for a CrCl of 10-30 include 40-56 h after completion of infusion. Further examples include 40, 41, 42, 43, 44, 45, 46, 47, 48, 49, 50, 51, 52, 53, 54, 55, to 56 h.

In another example, one implementation provides a method of initiating or escalating sotalol therapy, comprising: administering to a patient in need thereof an IV and oral dosage of sotalol selected by a physician, wherein the patient's physician selects the IV and oral dosages based on the patient's CrCl as defined in Table 1.

Exemplary Dofetilide Protocols

Any of the dosing protocols disclosed herein can be used with the medical device and/or system provided herein. Illustrated next are various protocols for administering anti-arrhythmics, such as dofetilide, but other anti-arrhythmics can be substituted, and depending on the particular anti-arrhythmic (such as for sotalol, amiodarone, ibutilide, dronedarone, procainamide, flecainide, or propafenone), the dose and/or rate of administration can be adjusted up or down to be commensurate with the pharmacokinetics of the particular drug being administered.

For example, dofetilide dosing protocols can include methods such as administering dofetilide to a subject/patient; wherein the dofetilide is administered: as a loading dose, intravenously, over a period of about 1 hour; and as a first IV or oral maintenance dose, in an amount of 125 μg, 250 μg, or 500 μg.

Additional dofetilide dosing protocols include a method of intravenously administering dofetilide, comprising: administering an IV loading dosage of dofetilide to a subject over a period of about 1 hour in the range of ±50% of a target oral dose; wherein the IV dosage is administered while the subject is in a facility capable of providing cardiac resuscitation and electrocardiographic monitoring.

Yet further methods include a method of intravenously administering dofetilide, comprising: administering an IV loading dosage of dofetilide to a subject over a period of about 10 minutes in the range of ±50% of a target oral dose; wherein the IV dosage is administered while the subject is in a facility capable of providing cardiac resuscitation and electrocardiographic monitoring.

Further, for example, dofetilide dosing methods include a method of intravenously administering dofetilide, comprising: administering an IV loading dosage of dofetilide to a subject over a period of about 1-5 hours, such as over 1 hour, 2 hours, 3 hours, 4 hours or 5 hours, such as in the range of ±50% of a target oral dose; and wherein the IV loading dosage and period are selected to enable maximum serum concentration of the subject within 24 hours of the start of administering the IV loading dose and within a range of +25% of a steady state Cₘₐₓ predicted for the subject for an oral dosing protocol of 125 Kg, 250 Kg or 500 Kg dofetilide.

### Example 1

An example dofetilide or anti-arrhythmic treatment protocol for a patient experiencing a ventricular conduction abnormality, such as atrial flutter, is described herein. The male patient, age 60, is admitted to the hospital to initiate anti-arrhythmic, such as dofetilide, treatment. The patient's creatinine clearance is measured/determined to be >60 mL/min. The patient is connected to an electrocardiograph, and treatment is initiated with an IV loading dose of dofetilide in an amount of up to 3.5 μg/kg infused over up to 1 hour. The patient's QT is measured/determined prior to start of the IV and is monitored every 15 minutes during the IV loading dose administration along with heart rate (HR) and blood pressure (BP). The patient's QTc is calculated from the QT measurement. If the patient's baseline (i.e., prior to dofetilide administration) QT interval or baseline QTc is greater than 500 msec (in patients with ventricular conduction abnormalities), then dofetilide is contraindicated. Measure every 15 min. during the IV loading dose, and if the QT or QTc increases by greater than 20% of the patient's baseline QT or baseline QTc (or is greater than 550 msec), then the IV dofetilide is discontinued or a subsequent lower dose is considered or administered. If after any subsequent IV or oral dose the QT or QTc increases to greater than 550 msec, then dofetilide is discontinued.

Immediately or one hour after completion of the loading dose, the patient receives a maintenance dose of dofetilide delivered by way of a second IV infusion of up to 2.5 μg/kg over 5 hours. The patient's QT or QTc is monitored every hour during the administration of the IV maintenance dose. If during or after the IV maintenance dose the patient's QT or QTc is greater than 550 msec or if the QT or QTc increases by greater than 20% of the patient's baseline QT or QTc, then the IV maintenance dose is reduced and subsequent IV maintenance doses are delivered at approximately 1.25 μg/kg over up to 5 hours.

For the situation where QT or QTc is within an acceptable range, one or more subsequent IV or oral maintenance doses, selected from a 2.5 μg/kg IV infusion over up to 5 hours or a 500 μg oral dose, are repeated every 12 hours until Cmax ss is reached and the patient can be released to continue dofetilide treatment orally outside the hospital. For the situation where (i) the patient's QT or QTc after administration of the first IV maintenance dose is greater than 550 msec, (ii) the patient's subsequent dose is reduced, and (iii) the QT or QTc for the subsequent reduced maintenance dose (or any subsequent maintenance dose) is observed to be greater than 550 msec, additional subsequent maintenance doses can be further reduced to a 0.5-0.9 μg/kg IV infusion over 5 hours, and/or given over a longer infusion time, and/or reduced to a 125 μg oral dose, or dofetilide treatment is discontinued.

### Example 2

An anti-arrhythmic, such as dofetilide, treatment protocol for a patient experiencing a ventricular conduction abnormality, such as atrial flutter, is described herein. The male patient, age 60, is admitted to the hospital to initiate treatment. The patient's creatinine clearance is determined to be between 40 and 60 mL/min. The patient is connected to an electrocardiograph, and an initial (or baseline) QT interval is determined to be not greater than 500 msec. Treatment is initiated with an IV loading dose of up to 1.6 m/kg infused over up to 1 hour. The loading dose may be administered by way of several smaller IV doses. The patient's QT interval is measured every 15 minutes during the IV loading dose administration along with HR and BP.

During or following administration of the IV loading dose, the patient's QT interval is determined to be greater than 550 msec. A reduced maintenance dose is administered to the patient up to 6 hours after completion of the IV loading dose or dofetilide administration is discontinued. The reduced maintenance dose is administered as an oral dose of 125 The patient's QT interval is monitored following administration of the oral maintenance dose. The patient's QT interval no longer exceeds 550 msec and the AQTc is not greater than 20% of the patient's initial QTc. Subsequent oral maintenance doses of 125 μg are given every 12 to 48 hours.

### Example 3

An example anti-arrhythmic/dofetilide treatment protocol for a patient experiencing a ventricular conduction abnormality, such as atrial flutter, is described herein. The male patient, age 60, is admitted to the hospital to initiate treatment. The patient's creatinine clearance is determined to be between 20 and 40 mL/min. The patient is connected to an electrocardiograph, and an initial QT interval is determined to be not greater than 500 msec. Treatment is initiated with an IV loading dose of up to 1.6m/kg infused over up to 1 hour. The loading dose may be administered by way of several smaller IV doses. The patient's QT interval is measured every 15 minutes during the IV loading dose administration along with HR and BP.

During and following administration of the loading dose, the patient's QT interval is determined to be not greater than 550 msec and AQTc is not greater than 20% of the patient's initial QTc. A maintenance dose is administered to the patient up to 6 hours after completion of the IV loading dose. The maintenance dose is administered as an oral dose of 125 μg. The patient's QT interval is monitored following administration of the oral maintenance dose. The patient's QT interval still does not exceed 550 msec and is not greater than 20% of the patient's initial QTc. Subsequent oral maintenance doses of 125 μg are given every 12 to 48 hours.

### Example 4

An example anti-arrhythmic/dofetilide treatment protocol for a patient experiencing a ventricular conduction abnormality, such as atrial flutter, is described herein. The male patient, age 60, is admitted to the hospital to initiate treatment. The patient's creatinine clearance is determined to be >60 mL/min. The patient is connected to an electrocardiograph, and an initial QT interval is determined. Treatment is initiated with an IV loading dose based on an oral target dose of 500 μg, which is administered in an amount of up to 3 μg/kg and is infused over up to 1 hour. The patient's QT interval is measured every 15 minutes during the IV loading dose administration along with HR and BP.

During and following administration of the loading dose, the patient's QT interval is determined to be not greater than 550 msec and the AQTc is not greater than 20% of the patient's initial QTc. A maintenance dose is administered to the patient as an IV dose of up to 3 μg/kg. The IV maintenance dose is administered over a time period of up to 60 minutes. The patient's QT interval is monitored during and following administration of the IV maintenance dose. The patient's QT interval still does not exceed 550 msec and the AQTc is not greater than 20% of the patient's initial QTc. Subsequent oral maintenance doses of 500 μg are given every 12 to 48 hours.

### Example 5

An example anti-arrhythmic/dofetilide treatment protocol for a patient experiencing a ventricular conduction abnormality, such as atrial flutter, is described herein. The male patient, age 60, is admitted to the hospital to initiate treatment. The patient's creatinine clearance is determined to be between 40 and 60 mL/min. The patient is connected to an electrocardiograph, and an initial QT interval is determined. Treatment is initiated with an IV loading dose based on an oral target of 250 μg, which is administered in an amount of up to 1.6 μg/kg and is infused over about 1 hour. The patient's QT interval is measured every 15 minutes during the IV loading dose administration along with HR and BP.

During and following administration of the loading dose, the patient's QT interval is determined to be not greater than 550 msec and the AQTc is not greater than 20% of the patient's initial QTc. A maintenance dose is administered to the patient as an IV dose of up to 1.6 μg/kg. The IV dose is administered over a time period of up to 60 minutes. The patient's QT interval is monitored during and following administration of the IV maintenance dose. The patient's QT interval still does not exceed 550 msec and the AQTc is not greater than 20% of the patient's initial QTc. Subsequent IV maintenance doses of up to 1.6 μg/kg are given every 12 to 48 hours.

### Example 6

An example anti-arrhythmic/dofetilide treatment protocol for a patient experiencing a ventricular conduction abnormality, such as atrial flutter, is described herein. The male patient, age 60, is admitted to the hospital to initiate treatment. The patient's creatinine clearance is determined to be between 20 and 40 mL/min. The patient is connected to an electrocardiograph, and an initial QT interval is determined. Treatment is initiated with an IV loading dose based on an oral target of 125 μg, which is administered in an amount of up to 0.811 g/kg and is infused over about 1 hour. The patient's QT interval is measured every 15 minutes during the IV loading dose administration along with HR and BP.

During and following administration of the IV loading dose, the patient's QT interval is determined to be not greater than 550 msec and the AQTc is not greater than 20% of the patient's initial QTc. A maintenance dose is administered to the patient as an IV dose of up to 0.8 μg/kg. The IV maintenance dose is administered over a time period of up to 60 minutes. The patient's QT interval is monitored during and following administration of the IV maintenance dose. The patient's QT interval still does not exceed 550 msec and the AQTc is not greater than 20% of the patient's initial QTc. Subsequent IV maintenance doses of up to 0.8 μg/kg are given every 12 to 48 hours.

### Example 7

An example anti-arrhythmic/dofetilide treatment protocol for a patient experiencing a ventricular conduction abnormality, such as atrial flutter, is described herein. The female patient, age 60, is admitted to the hospital to initiate treatment. The patient's creatinine clearance is determined to be >60 mL/min. The patient is connected to an electrocardiograph, and an initial QT interval is determined to be in an acceptable range. Treatment is initiated with an IV loading dose in the range of 1.6-3 μg/kg. The patient's QT interval is measured every 15 minutes during the IV loading dose administration along with HR and BP.

During or following administration of the IV loading dose, the patient's QT interval is determined to be not greater than 550 msec, but the AQTc is greater than 20% of the patient's initial QTc. A maintenance dose is administered to the patient in a reduced amount as an oral dose of 250 The patient's QT interval is monitored following administration of the first oral maintenance dose. The patient's QT interval still does not exceed 550 msec and the AQTc is no longer greater than 20% of the patient's initial QTc. Subsequent oral maintenance doses of 250 μg are given every 12 to 48 hours.

### Example 8

An example anti-arrhythmic/dofetilide treatment protocol for a patient experiencing a ventricular conduction abnormality, such as atrial flutter, is described herein. The male patient, age 50, is admitted to the hospital to initiate treatment. The patient's creatinine clearance is determined to be between 40 and 60 mL/min. The patient is connected to an electrocardiograph, and an initial QT interval is determined. Treatment is initiated with an IV loading dose of 0.8-1.6 μg/kg. The patient's QT interval is measured every 15 minutes during the IV loading dose administration along with HR and BP.

During and following administration of the IV loading dose, the patient's QT interval is determined to be not greater than 550 msec and the AQTc is not greater than 20% of the patient's initial QTc. A first maintenance dose is administered to the patient as an oral dose of 250 The patient's QT interval is monitored following administration of the first oral maintenance dose. The patient's QT interval is determined to be less than 550 msec and the AQTc is not greater than 20% of the patient's initial QTc. Oral maintenance doses of 250 μg are given every 12-48 hours.

### Example 9

An example anti-arrhythmic/dofetilide treatment protocol for a patient experiencing a ventricular conduction abnormality, such as atrial flutter, is described herein. The male patient, age 40, is admitted to the hospital to initiate treatment. The patient's creatinine clearance is determined to be between 20 and 40 mL/min. The patient is connected to an electrocardiograph, and an initial QT interval is determined. Treatment is initiated with an IV loading dose of 0.1-0.8 μg/kg. The patient's QT interval is measured every 15 minutes during the IV loading dose administration along with HR and BP.

During and following administration of the IV loading dose, the patient's QT interval is not greater than 550 msec and the AQTc is not greater than 20% of the patient's initial QTc. A first maintenance dose is administered to the patient as an oral dose of 125 The patient's QT interval is monitored following administration of the first oral maintenance dose. The patient's QT interval is determined to be less than 550 msec and the AQTc is not greater than 20% of the patient's initial QTc. Subsequent oral maintenance doses of 125 μg are given every 12 to 48 hours.

### Example 10

An example anti-arrhythmic/dofetilide treatment protocol for a patient experiencing a ventricular conduction abnormality, such as atrial flutter, is described herein. The female patient, age 40, is admitted to the hospital to initiate treatment. The patient's creatinine clearance is determined to be between >60 mL/min. The patient is connected to an electrocardiograph, and an initial QT interval is determined. Treatment is initiated with an IV loading dose of about 3 μg/kg. The patient's QT interval is measured every 15 minutes during the IV loading dose administration along with HR and BP.

During or following administration of the IV loading dose, the patient's QT interval is determined to be greater than 550 msec. A first maintenance dose is administered to the patient as an oral dose in the reduced amount of 250 The patient's QT interval is monitored following administration of the first oral maintenance dose. The patient's QT interval is still determined to be greater than 550 msec. Subsequent oral maintenance doses of 125 μg are given every 12 to 48 hours or dofetilide administration is discontinued.

### Example 11

An example anti-arrhythmic/dofetilide treatment protocol for a patient experiencing a ventricular conduction abnormality, such as atrial flutter, is described herein. The female patient, age 40, is admitted to the hospital to initiate treatment. The patient's creatinine clearance is determined to be between 40 to 60 mL/min. The patient is connected to an electrocardiograph, and an initial QT interval is determined. Treatment is initiated with an IV loading dose of about 1.6 μg/kg. The patient's QT interval is measured every 15 minutes during the IV loading dose administration along with HR and BP.

During and following administration of the IV loading dose, the patient's QT interval is determined to be 550 msec or less and the AQTc is not greater than 20% of the patient's initial QTc. A first maintenance dose is administered to the patient as an oral dose of 250 The patient's QT interval is monitored following administration of the first oral maintenance dose. The patient's QT interval is still determined to be less than 550 msec and the AQTc is not greater than 20% of the patient's initial QTc. The physician would like to further lengthen the patient's QTc, therefore subsequent oral maintenance doses of 500 μg are given every 12 to 48 hours.

### Example 12

An example anti-arrhythmic/dofetilide treatment protocol for a patient experiencing a ventricular conduction abnormality, such as atrial flutter, is described herein. The male patient, age 40, is admitted to the hospital to initiate treatment. The patient's creatinine clearance is determined to be between 20 and 40 mL/min. The patient is connected to an electrocardiograph, and an initial QT interval is determined. Treatment is initiated with an IV loading dose in an amount ranging from 0.1-0.8 μg/kg. The patient's QT interval is measured every 15 minutes during the IV loading dose administration along with HR and BP.

During and following administration of the IV loading dose, the patient's QT interval is not greater than 550 msec and the AQTc is not greater than 20% of the patient's initial QTc. A first maintenance dose is administered to the patient as an oral dose of 125 The patient's QT interval is monitored following administration of the first oral maintenance dose and the patient's QT interval is determined to be not greater than 550 msec, and the AQTc is not greater than 20% of the patient's initial QTc. A second maintenance dose is administered to the patient as an oral dose of 125 μg and is given 12 to 24 hours after the first maintenance dose. The patient's QTc interval is monitored following administration of the second oral maintenance dose and the patient's QTc interval is determined to be not greater than 550 msec, but the AQTc is greater than 20% of the patient's initial QTc (or the patient's QTc interval is determined to be greater than 550 msec, but the AQTc is not greater than 20% of the patient's initial QTc, or both are outside of the acceptable range). Subsequent oral maintenance doses of 125 μg are given farther apart and every 24 to 48 hours.

### Example 13

An example anti-arrhythmic/dofetilide treatment protocol for a patient currently being administered oral dofetilide for treatment of a ventricular conduction abnormality, such as atrial flutter, is described herein. The male patient, age 60, is admitted to the hospital to uptitrate dofetilide treatment from a 250 μg BID protocol to a 500 μg BID protocol. The patient's creatinine clearance is determined to be >60 mL/min. The patient is connected to an electrocardiograph, and an initial QTc interval is determined. An IV loading dose is administered to the patient based on an oral target dose of 500 which is administered in an amount of up to 3 μg/kg and is infused over up to 1 hour. The patient's QTc interval is measured every 15 minutes during the IV loading dose administration along with HR and BP.

During and following administration of the loading dose, the patient's QTc interval is determined not to exceed 550 msec and the AQTc is not greater than 20% of the patient's initial QTc. A maintenance dose is administered to the patient as an IV dose of up to 3 μg/kg. The IV maintenance dose is administered over a time period of up to 60 minutes. The patient's QTc interval is monitored during and following administration of the IV maintenance dose. The patient's QTc interval still does not exceed 550 msec and the AQTc is not greater than 20% of the patient's initial QTc. Subsequent oral maintenance doses of 500 μg are given every 12 hours.

### Example 14

Antiarrhythmic Drug Administration System

A patient in need of antiarrhythmic drug treatment or therapy, for example sotalol hydrochloride, is admitted for initiation or escalation of the drug while under QTc monitoring. Patient data are inputted into the medical device for determining dosing of the antiarrhythmic drug, including a creatinine clearance or creatinine level of the patient, optionally a start time for the infusion to begin, whether the patient is being initiated or escalated on the drug, and the target oral dose for the patient. For example, for initiating a patient on an antiarrhythmic drug, the target oral or IV dose is input into the medical device. For escalating a patient from one current oral or IV dose to a second higher oral or IV dose, escalation is indicated as well as the target oral dose. For example, if escalation is indicated with a target oral dose of 120 mg of sotalol hydrochloride, then the current oral dose would be understood to be 80 mg. Likewise, if escalation is indicated with a target oral dose of 160 mg, then the current oral dose would be understood to be 120 mg. In embodiments, the patient is optionally connected to a patient monitoring device configured to obtain at least heart rate and/or QT interval and/or QTc data and/or creatinine clearance. In embodiments, the patient monitor is connected with the medical device for determining dosing, such that the heart rate and/or QT interval and/or QTc data, and/or patient creatinine clearance, can be automatically input, or a health professional can input the information into the medical device manually from any one or more patient monitoring device or database if desired. Based on the inputted infusion start time, whether the patient is being escalated or initiated, the patient's creatinine clearance, the target oral dose, and optionally heart rate and/or QT interval and/or QTc data, the medical device for determining dosing of the antiarrhythmic drug determines a treatment plan based on stored dosage criteria/protocols. In embodiments, the stored dosage criteria or storage of any patient/subject data or storage of software, the app, or computer-executable instructions can be stored in a different location (than on the medical device) and made available or accessible to the medical device (such as through networking and/or cloud computing). The treatment plan can be approved by a physician remotely or on a system interface, such as from a smartphone. A first IV loading dose of antiarrhythmic drug is administered to the patient based on the treatment plan determined by the medical device for determining the dosing of the antiarrhythmic drug. The administering can be performed/controlled directly or indirectly by an administration system, for example, by controlling an infusion pump to deliver the antiarrhythmic drug to the patient. The device/system can also be configured to provide instructions to a health professional regarding the treatment plan such that the health professional can control the infusion pump to administer the antiarrhythmic drug to the patient according to the treatment plan. Included in the outputs of the treatment plan are the amount of the IV dose, the time of the first oral dose and the oral dosing interval or time or subsequent oral dose(s) if any. The dose amount and administration time are provided by the treatment plan and are based on creatine clearance or liver clearance capability. In the case of sotalol hydrochloride, the treatment plan is based on the patient's creatinine clearance or creatinine level, whether the patient is being initiated or escalated on the drug, the target oral dose, and the projected start time for the protocol to begin, i.e., the start time for IV infusion. The time for oral dosing to begin is based on the patient's creatinine clearance and the start time of the infusion. The oral dosing interval is based on the patient's creatinine clearance or creatinine level, and a start time for oral doses subsequent to the first oral dose is based on the start time of the first oral dose. If a different start time than the initially projected start time for infusion is needed, then the dosing protocol can be recalculated based on a new input of an actual start time for infusion and/or a new projected start time for infusion. If more than one IV dosing option is appropriate, then a choice of IV dosing options can be presented/outputted.

In embodiments, the patient monitor can obtain heart rate and/or QT intervals either continuously or at time periods established by the treatment plan according to the dosing protocol. Based on the QT interval response to the antiarrhythmic drug being administered, additional IV doses of antiarrhythmic drug are administered and/or oral doses of antiarrhythmic drug are administered/instructed by the system. Based on the QT interval response, the IV and/or oral dosages are adjusted accordingly, for example, the dosage amount can be decreased, increased or halted. In embodiments, the administration system is configured to halt treatment, or provide instructions for halting treatment, and notify a system user, for example by way of an alarm or alert such as a text alert, if the patient's heart rate or QT interval deviate from predetermined accepted values. The administration system is additionally configured to update the treatment plan based on the QT interval response during and after administration of each dose of antiarrhythmic drug. If the treatment plan is updated by the system, the system can prompt a user for approval prior to further administration of antiarrhythmic drug.

### Example 15

### Example Sotalol Protocol

Dosing criteria for the device and/or system of Example 14 above can include criteria for infusing a loading dose of sotalol for a period of one hour, as well as criteria for monitoring a patient's QTc at 15 minute intervals during the infusion. If the baseline QTc is >450 ms (JT >330 ms if QRS over 100 ms), sotalol is not recommended.

Monitoring of QTc is continued around Tₘₐₓ (2 to 4 hours post-dose) following the first oral dose (in all patients) and second oral dose (in patients with CrCl ≥60 mL/min).

If the QTc prolongs to ≥500 ms or increases 20% from baseline when initiating for an oral dose of 80 mg, the dosing criteria involves discontinuing the drug; if initiating for an oral dose of 120 mg the dosing criteria involves discontinuing the drug and considering a lower dose. If re-initiation at a lower dose of 80 mg is desired, the dosing criteria involves waiting at least 1 day (in patients with CrCl ≥60 mL/min), or at least 3 days (in patients with CrCl ≥30 to <60 mL/min), or 7 days (in patients with CrCl ≥10 to <30 mL/min).

The dosing criteria of the administration system comprises amounts of various intravenous loading doses, the dosing interval(s) for oral or IV maintenance administration, and the minimum delay (if any) between the end of the infusion and the first oral or IV maintenance dose, which depend on the target oral or IV dose and creatinine clearance; see Table 1.

### Example 16

Sotalol Administration System

A specific example of a medical device and/or system employing the particular features of Examples 14-15 is provided as follows. A patient is identified for whom initiation or escalation of sotalol is desired while under QTc monitoring. For initiating a patient on sotalol, the target oral sotalol dose is input into the administration system. For escalating a patient from a current/previous oral sotalol dose (e.g., 80 mg or 120 mg) to a second higher oral sotalol dose (e.g., respectively 120 mg or 160 mg), escalation is input into the administration system along with the target oral sotalol dose. Based on the inputted patient data, the medical device for determining dosing of the antiarrhythmic drug determines a treatment plan based on dosage criteria/protocols.

The dosage criteria/protocols comprise the following rules relating to determining the appropriate IV loading dose for a particular patient:

Dosing based on administering a loading dose of sotalol hydrochloride over a period of 1 hour, wherein the IV loading dosage criteria is selected from dosages I(a)-IV(d):
I for patients having a creatinine clearance (CrCl) of >90 mL/min;
(a) 60 mg for a patient naïve to sotalol and having a target of 80 mg;
(b) 90 mg for a patient naïve to sotalol and having a target of 120 mg;
(c) 75 mg for a patient previously receiving 80 mg of sotalol;
(d) 90 mg for a patient previously receiving 120 mg of sotalol;
II for patients having a creatinine clearance (CrCl) of 60 to 90 mL/min;
(a) 82.5 mg for a patient naïve to sotalol and having a target of 80 mg;
(b) 125 mg for a patient naïve to sotalol and having a target of 120 mg;
(c) 82.5 mg for a patient previously receiving 80 mg of sotalol;
(d) 105 mg for a patient previously receiving 120 mg of sotalol;
III for patients having a creatinine clearance (CrCl) of 30 to 60 mL/min;
(a) 75 mg for a patient naïve to sotalol and having a target of 80 mg;
(b) 112.5 mg for a patient naïve to sotalol and having a target of 120 mg;
(c) 82.5 mg for a patient previously receiving 80 mg of sotalol;
(d) 105 mg for a patient previously receiving 120 mg of sotalol;
IV for patients having a creatinine clearance (CrCl) of 10 to 30 mL/min;
(a) 75 mg for a patient naïve to sotalol and having a target of 80 mg;
(b) 112.5 mg for a patient naïve to sotalol and having a target of 120 mg;
(c) 82.5 mg for a patient previously receiving 80 mg of sotalol;
(d) 105 mg for a patient previously receiving 120 mg of sotalol.

A first IV loading dose of sotalol is administered to the patient based on the above dosing criteria applicable to the patient. For example, the IV loading dose according to protocol I(a) would be determined by the device or system as an appropriate loading dose for a patient who is naïve to sotalol, with a target oral dose of 80 mg and having a creatinine clearance in the range of >90 mL/min. The administering can be performed/controlled directly or indirectly by any component of the administration system, for example, by controlling an infusion pump to deliver the antiarrhythmic drug to the patient. The system can also be configured to provide instructions to a health professional regarding the treatment plan such that the health professional can control the infusion pump to administer the antiarrhythmic drug to the patient according to the treatment plan.

In embodiments, optionally the patient monitor continues to obtain heart rate and/or QT intervals either continuously or at time periods established by the treatment plan according to the dosing protocol. If the QTc prolongs to ≥500 ms or increases 20% from baseline when initiating for an oral dose of 80 mg, the dosing criteria involves discontinuing the drug; if initiating for an oral dose of 120 mg the dosing criteria involves discontinuing the drug and considering a lower dose. If re-initiation at a lower dose of 80 mg is desired, the dosing criteria involves waiting at least 1 day (in patients with CrCl ≥60 mL/min), or at least 3 days (in patients with CrCl ≥30 to <60 mL/min), or 7 days (in patients with CrCl ≥10 to <30 mL/min).

Based on the QT interval response to the antiarrhythmic drug being administered, oral doses of antiarrhythmic drug are administered/instructed by the system according to dosing criteria adhering to the following rules:

Dosing based on orally administering sotalol hydrochloride to the patient, wherein the dosing criteria of the administration system are selected from the following dosage/intervals selected from (I)-(IX):
I 80 mg BID, starting 4 hours after completion of IV doses I(a) or II(a);
II 120 mg BID, starting 4 hours after completion of IV doses I(b), I(c), II(b), or II(c);
III 160 mg BID, starting 4 hours after completion of IV doses I(d) or II(d);
IV 80 mg QD, starting 6 hours after completion of IV dose III(a);
V 120 mg QD, starting 6 hours after completion of IV doses III(b) or III(c);
VI 160 mg QD, starting 6 hours after completion of IV dose III(d);
VII 80 mg at a 48 h interval, starting 12 hours after completion of IV dose IV(a);
VIII 120 mg at a 48 h interval, starting 12 hours after completion of IV doses IV(b) or IV(c);
IX 160 mg at a 48 h interval, starting 12 hours after completion of IV dose IV(d).

Based on the QT interval response, the medical device or system can indicate that IV and/or oral dosages should be adjusted accordingly, for example, the dosage amount can be decreased, increased or halted. In embodiments, the administration system is configured to halt treatment, or provide instructions for halting treatment, and notify a system user, for example by way of an alarm or alert such as a text alert, if the patient's heart rate or QT interval deviate from predetermined accepted values. The administration system can also be configured to update the treatment plan based on the QT interval response during and/or after administration of each dose of antiarrhythmic drug.

### Example 17

Dofetilide Administration System

A specific example of a medical device and/or system is provided as follows. A patient is identified for whom dofetilide treatment is desired while under QTc monitoring. The target maintenance dose (IV or oral) of dofetilide is input into the administration system. Based on the inputted patient data, the medical device for determining dosing of the antiarrhythmic drug determines a treatment plan based on dosage criteria/protocols.

The dosage criteria/protocols comprise the following rules relating to determining the appropriate IV loading dose of dofetilide for a particular patient:

Dosing based on administering a loading dose of dofetilide, such as over a period of 1 hour, wherein the IV loading dosage criteria is selected from dosages
I. 250 mcg-750 mcg, such as for patients having a creatinine clearance (CrCl) of >60 mL/min, a target oral dose of 500 mcg;
II. 125 mcg-375 mcg, such as for patients having a creatinine clearance (CrCl) of 40 to 60 mL/min;
III. 62.5 mcg-187.5 mcg, such as for patients having a creatinine clearance (CrCl) of 20 to <40 mL/min.

A first IV loading dose of dofetilide is administered to the patient based on the above dosing criteria applicable to the patient. For example, the IV loading dose according to protocol I would be determined by the device or system as an appropriate loading dose for a patient with a target oral dose of 500 mcg and having a creatinine clearance in the range of >60 mL/min. The administering can be performed/controlled directly or indirectly by any component of the administration system, for example, by controlling an infusion pump to deliver the antiarrhythmic drug (e.g., dofetilide) to the patient. The system can also be configured to provide instructions to a health professional regarding the treatment plan such that the health professional can control the infusion pump to administer the antiarrhythmic drug to the patient according to the treatment plan.

In embodiments, optionally the patient monitor continues to obtain heart rate and/or QT intervals either continuously or at time periods established by the treatment plan according to the dosing protocol. If the QTc prolongs to ≥500 ms (550 msec in patients with ventricular conduction abnormalities) or increases more than 15% from baseline when initiating for an oral dose of 500 mcg, the dosing criteria involves discontinuing the drug and considering a lower dose (such as 250 mcg); if initiating for an oral dose of 250 mcg the dosing criteria involves discontinuing the drug and considering a lower dose (such as 125 mcg); if initiating for an oral dose of 125 mcg the dosing criteria involves discontinuing the drug and considering a decreased dosing frequency (e.g., decreasing dose frequency from twice daily administration to once daily administration).

Based on the QT interval response, the medical device or system can indicate that IV and/or oral dosages should be adjusted accordingly, for example, the dosage amount can be decreased, increased or halted. In embodiments, the administration system is configured to halt treatment, or provide instructions for halting treatment, and notify a system user, for example by way of an alarm or alert such as a text alert, if the patient's heart rate or QT interval deviate from predetermined accepted values. The administration system can also be configured to update the treatment plan based on the QT interval response during and/or after administration of each dose of antiarrhythmic drug.

Based on the QT interval response to the antiarrhythmic drug being administered, oral doses of antiarrhythmic drug are administered/instructed by the system according to dosing criteria adhering to the following rules:

Dosing based on orally administering dofetilide to the patient, wherein the dosing criteria of the administration system are selected from the following dosage/intervals selected from (I)-(III):
I. 500 mcg BID, starting up to 4 hours after completion of IV dose I;
II. 250 mcg BID, starting up to 4 hours after completion of IV dose II;
III. 125 mcg BID, starting up to 4 hours after completion of IV dose III.

Based on the QT interval response, the medical device or system can indicate that IV and/or oral dosages should be adjusted accordingly, for example, the dosage amount can be decreased, increased or halted. In embodiments, the administration system is configured to halt treatment, or provide instructions for halting treatment, and notify a system user, for example by way of an alarm or alert such as a text alert, if the patient's heart rate or QT interval deviate from predetermined accepted values. The administration system can also be configured to update the treatment plan based on the QT interval response during and/or after administration of each dose of antiarrhythmic drug.

### Example 18

Another implementation of a method is shown in FIG. 13. The method can start with prompting or receiving input of a patient's creatinine clearance 102. For antiarrhythmic drugs where liver clearance is an issue, the health professional can instead or in addition be prompted to enter a value relating to the patient's liver clearance ability or the creatinine clearance can be presented by the user interface by way of a drop down list to be selected. Based on the input of the patient's creatinine clearance or liver clearance capability, an oral and/or IV dosing interval is determined, and the oral and/or IV dosing interval is outputted 103. For antiarrhythmic drugs that are administered intravenously, an IV dosing schedule/interval will be outputted. If the inputted creatinine clearance is greater than 60 mL/min, then for dofetilide a dosing interval of up to 12 hours is outputted 104 (e.g., administration of subsequent oral and/or IV doses is indicated to be at 12 hour intervals from the first maintenance dose, such as from the first oral dose). For a creatinine clearance input of within the range of 40 to 60 mL/min, then a dosing interval for dofetilide of up to 24 hours is outputted 105 (e.g., administration of subsequent (e.g., maintenance) oral and/or IV doses is indicated to be at 24 hour intervals from the first maintenance dose, such as from the first oral dose), or a choice of dosing intervals can be provided if more than one option applies. For a creatinine clearance input of within the range of 20 to <40 mL/min, then a dosing interval for dofetilide of up to 48 hours is outputted 106 (e.g., administration of subsequent oral and/or IV doses is indicated to be at 48 hour intervals from the first maintenance dose, such as from the first oral dose), or a choice of interval options is presented.

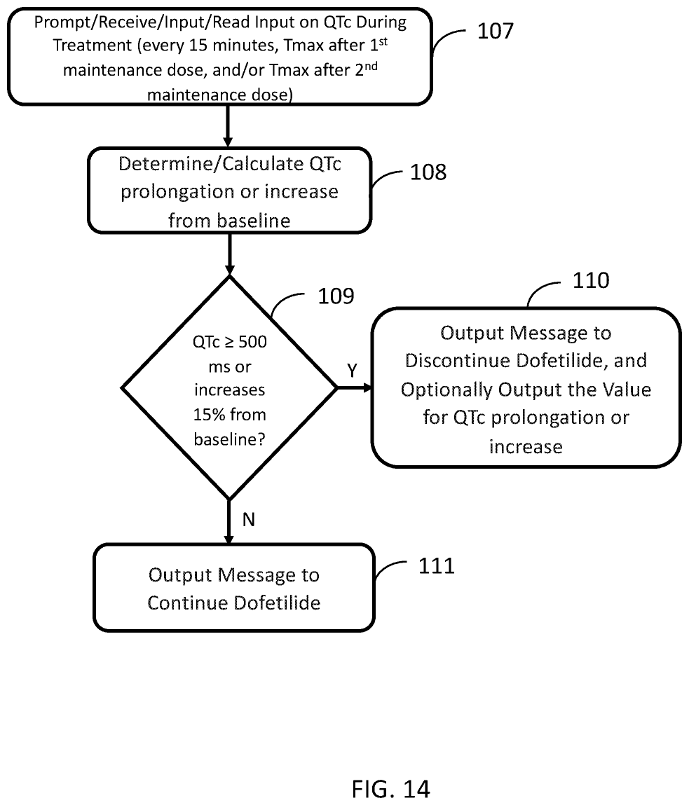

Another implementation of a method is shown in FIG. 14. For dofetilide, as an example, a health professional is first prompted to input patient QTc during dofetilide treatment 107. The QTc can be measured and then inputted at 15 minute intervals, such as at or around Tₘₐₓ (i.e., the time expected to reach Cₘₐₓ) after a loading dose, and/or at or around Tₘₐₓ after one or more maintenance dose. Then, QTc prolongation or increase from baseline are determined or calculated based on the inputted QTc 108. Then, a determination is made as to whether QTc is exceeds 500 ms or increases more than 15% from baseline 109. If QTc is exceeds 500 ms or increases more than 15% from baseline, then a message to discontinue dofetilide is outputted 110; the QTc value and/or percentage increase from baseline value may also be optionally outputted. If QTc does not exceed 500 ms or increase more than 15% from baseline, then a message to continue dofetilide is outputted 111.

Optionally, after an IV loading dose of dofetilide or after a first oral dose of dofetilide, if the increase in QTc is up to 15% from baseline, the current target dose is continued. Optionally, after an IV loading dose of dofetilide or a first oral dose of dofetilide, if the increase in QTc is greater than 15% from baseline or is >500 ms, the target of dofetilide is decreased, such as by about ½ (e.g., an initial target oral dose of 500 mcg would be reduced to 250 mcg).

The present disclosure has described particular implementations having various features. In light of the disclosure provided herein, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A treatment management application configured to perform a set of operations comprising:
(i) reading, prompting input of and/or accepting the following as inputs:
a projected start time for a dofetilide infusion;
a creatinine clearance (CrCl) or creatinine level of a subject; and
an amount of an oral or IV maintenance dosage of dofetilide for the subject; and
(ii) executing computer executable instructions to derive from a set of rules a dofetilide dosing protocol for the subject based on the inputs, the dosing protocol comprising:
one or more amount of the dofetilide for infusion as a loading dose;
a time for a first oral or IV maintenance dose of dofetilide; and
optionally a time for a second oral or IV maintenance dose of dofetilide; and
(iii) outputting the dofetilide dosing protocol.

2. The treatment management application of claim 1, wherein the set of operations further comprises:
reading, prompting input of and/or accepting as an input an actual start time for the dofetilide infusion; and
outputting a revised dosing protocol with confirmed or updated times for the first maintenance dose and optionally the second maintenance dose.

3. The treatment management application of claim 1, wherein:
(a) 500 mcg is the maintenance dose, and the amount of the dofetilide loading dose is in the range of 250-750 mcg; or
(b) 250 mcg is the maintenance dose, and the amount of the dofetilide loading dose is in the range of 125-375 mcg; or
(c) 125 mcg is the maintenance dose, and the amount of the dofetilide loading dose is in the range of 62.5-187.5 mcg.

4. A method of using a treatment management application to provide a dofetilide dosing regimen for a patient, comprising:
a. measuring a creatinine clearance of the patient;
b. determining an amount of an oral dofetilide dose to administer to the patient;
c. inputting the patient's creatinine clearance and the amount of the oral dofetilide dose into a treatment management application to provide an output comprising an amount of an intravenous (IV) dose of dofetilide to administer to the patient;
d. administering the IV dose to the patient over a selected period of time; and
e. administering the oral dofetilide dose determined in step (b) to the patient after the IV dose has been administered, including optionally repeating one or more times the administering of the oral dofetilide dose at a selected dosing interval.

5. The method of claim 4, further comprising:
f. measuring a baseline QT_{c} interval for the patient;
g. determining one or more QT_{c} intervals after administration of the IV dose; and
h. if at least one of the one or more QT_{c} intervals determined in step (g) is above 500 msec or more than 15% greater than the baseline QT_{c} interval, discontinuing the administering of the IV dose of dofetilide, or the oral dofetilide dose, or one or more of the repeating of the administering of the oral dofetilide dose.

6. A method of providing a dofetilide dosing regimen for a patient, comprising:
a. measuring a creatinine clearance of the patient;
b. determining an amount of an oral dofetilide dose to administer to the patient;
c. inputting the patient's creatinine clearance and the amount of the oral dofetilide dose into a treatment management application to provide an output comprising an amount of an intravenous (IV) dose of dofetilide to administer to the patient;
d. administering the IV dose to the patient; and
e. administering the oral dofetilide dose determined in step (b) to the patient after the IV dose has been administered, including optionally repeating one or more times the administering of the oral dofetilide dose at a selected dosing interval.

7. The method of claim 6, further comprising:
f. measuring a baseline QT_{c} interval for the patient;
g. determining one or more QT_{c} intervals after administration of the IV dose; and
h. if at least one of the one or more QT_{c} intervals determined in step (g) is above 500 msec or more than 15% greater than the baseline QT_{c} interval, discontinuing the administering of the IV dose of dofetilide, or the oral dofetilide dose, or one or more of the repeating of the administering of the oral dofetilide dose.

8. The method of claim 6, further comprising:
f. measuring a baseline QT_{c} interval for the patient;
g. determining one or more QT_{c} intervals after administration of the IV dose; and
h. inputting the one or more QT_{c} intervals into the treatment management application to provide an output comprising:
an updated oral dofetilide dose to administer to the patient; and/or
an updated dosing interval;
i. administering the oral dofetilide dose to the patient according to the updated dosing interval, or administering the updated oral dofetilide dose to the patient according to the selected dosing interval or according to the updated dosing interval.

9. The method of claim 8, wherein if at least one of the one or more QT_{c} intervals is above 500 msec or more than 15% greater than the baseline QT_{c} interval, the updated oral dofetilide dose is lower than the oral dofetilide dose and/or the updated dosing interval is longer than the selected dosing interval.

10. The method of claim 9, wherein:
for an oral dofetilide dose of 500 μg, the updated oral dose is 250 μg; or
for an oral dofetilide dose of 250 μg, the updated oral dose is 125 μg.

11. The method of claim 9, wherein:
for an oral dofetilide dose of 125 μg with a selected dosing interval of 12 hours, the updated dosing interval is 24 hours.

12. The method of claim 6, wherein:
a creatinine clearance of the patient is >60 mL/min;
the IV dose is an amount in the range of 125 μg-625 μg;
the amount of the oral dofetilide dose is 500 μg; and
the administering of the oral dofetilide dose comprises repeating one or more times the administering of the oral dofetilide dose at a selected dosing interval of 12 hours from a previous oral dofetilide dose.

13. The method of claim 6, wherein the oral dofetilide dose is 250 μg.

14. The method of claim 6, wherein:
the creatinine clearance of the patient is in the range of 40-60 mL/min;
the IV dose is an amount in the range of 62.5 μg-312.5 μg; and
the administering of the oral dofetilide dose comprises repeating one or more times the administering of the oral dofetilide dose at a selected dosing interval of 12 hours from a previous oral dofetilide dose.

15. The method of claim 6, wherein the oral dofetilide dose is 125 μg.

16. The method of claim 6, wherein:
the patient has a creatinine clearance in the range of 20 to <40 mL/min;
the IV dose is an amount in the range of 31.25 μg-156.25 μg; and
the administering of the oral dofetilide dose comprises repeating one or more times the administering of the oral dofetilide dose at a selected dosing interval of 12-24 hours from a previous oral dofetilide dose.

17. The method of claim 6, further comprising inputting a projected start time for the IV dose into the treatment management application to provide an output comprising a start time for administering the oral dofetilide dose and optionally a start time for one or more of the repeating of the administering of the oral dofetilide dose.
